(19) United States
(12) Patent Application Publication
(10) Pub. No.: US 2006/0270669 A1
(43) Pub. Date: Nov. 30, 2006

(54) Method of preparing 3-cyano-quinolines and intermediates made thereby

(76) Inventors: Warren Chew, Outremont (CA); Maria Papamichelakis, Montreal (CA)

Correspondence Address: FITZPATRICK CELLA (WYETH), 30 ROCKEFELLER PLAZA, NEW YORK, NY, 10112-3800, US

(21) Appl. No.: 11/442,561

(22) Filed: May 25, 2006

Related U.S. Application Data
(60) Provisional application No. 60/684,683, filed on May 25, 2005.

(51) Int. Cl. A61K 31/5377; A61K 31/506; A61K 31/496; A61K 31/4709; C07D 413/02; C07D 403/02; C07D 215/38

(52) U.S. Cl. 514/232.5; 544/126; 544/333; 544/363; 546/153; 546/159; 514/256; 514/253.06; 514/312; 514/313

(57) ABSTRACT

The present invention relates to methods for preparing substituted 3-cyanoquinolines and intermediates obtained by the methods of the present invention. The methods of the invention comprise reacting an N-aryl-2-propanimide with phosphoryl chloride to produce the substituted 3-cyanoquinolines. The methods further comprise reacting arylamines, orthoformates and active methylenes to produce the N-aryl-2-propenamide.

This application claims the benefit of U.S. Provisional Application. No. 60/684,683, filed on May 25, 2005.

### BACKGROUND OF THE INVENTION

### Field of the Invention

The invention relates to a method for the preparation of substituted 3-cyanoquinolines. The 3-cyanoquinolines are made by two separate pathways which include the reaction of arylamines, orthoformates and active methylenes. Both pathways result in the production of N-aryl-2-propene derivatives.

Protein kinases are a class of enzymes that catalyze the transfer of a phosphate group from ATP to a tyrosine, serine, threonine, or histidine residue located on a protein substrate, many of which play a role in normal cell growth. Correspondingly, several growth factor receptor proteins function as protein tyrosine kinases (PTKs) to effect signaling and are known as receptor tyrosine kinases (RTKs).

The RTKs comprise one of the larger families of PTKs and have diverse biological activity. At present, at least nineteen distinct subfamilies of RTKs have been identified. One such subfamily is the “HER” family of RTKs, which includes EGFR (epithelial growth factor receptor), HER2, HER3 and HER4. It has been shown that under certain conditions, as a result of either mutation or over expression, these RTKs can become deregulated; the result of which is uncontrolled cell proliferation which can lead to tumor growth and cancer [Wilks, A. F., Adv. Cancer Res., 60, 43 (1993) and Parsons, J. T.; Parsons, S. J., Important Advances in Oncology, DeVita, V. T. Ed., J. B. Lippincott Co., Phila., 3 (1993)]. For example, over expression of the receptor kinase product of the erbB-2 oncogene has been associated with human breast and ovarian cancers [Slamon, D. J. et al., Science, 244, 707 (1989) and Science, 235, 177 (1987)]. In addition, deregulation of EGF-R kinase has been associated with epidermoid tumors [Reiss, M., et al., Cancer Res., 51, 6254 (1991)], breast tumors [Macias, A. et al., Anticancer Res., 7, 459 (1987)], and tumors involving other major organs [Gullick, W. J., Brit. Med. Bull., 47, 87 (1991)]. RTK inhibitors, therefore, have potential therapeutic value for the treatment of cancer and other diseases characterized by uncontrolled or abnormal cell growth. Accordingly, many recent studies have dealt with the development of specific RTK inhibitors as potential anti-cancer therapeutic agents [some recent reviews: Traxler, P., Exp. Opin. Ther. Patents, 8, 1599 (1998) and Bridges, A. J., Emerging Drugs, 3, 279 (1998)].

U.S. Pat. Nos. 6,002,008; 6,288,082; and 6,297,258, all of Wissner et al., describe such PTK, and particularly, RTK inhibitor compounds. The compounds of the Wissner et al. patents are all substituted 3-cyanoquinolines. The Wissner et al. patents are all incorporated herein by reference in their entirety.

Known methods of making quinolines utilize the thermal cyclization reaction. See, R. W. Sabnis and D. W. Rangnekar, J. Hetero. Chem. 29:65 (1992); N. C. Mehta and C. M. Desai, J. Ind. Chem. Soc. 55:193 (1978); H. Bredereck, F. Effenberger, H. Botsch and H. Rehn, Chem. Ber. 98:1081 (1965); J. Salon, V. Milita, N. Pronayova and J. Lesko, Monatsh. fur Chem. 131:293 (2000). Such thermal cyclization reactions require high temperatures (>240° C.) which limit their practicality for large scale production. In addition, such processes also require high dilution conditions resulting in decreased overall throughput and production. The yields in the thermal cyclization reaction are typically 50% or less but are variable and can range from 19-94%.

A microwave-assisted methodology for the production of quinolines from aromatic amines has also been described. See C. G. Dave and H. M. Joshipura, Ind. J. Chem. 41B:650 (2002).

Subsequent functionality of hydroxyquinolines typically requires chlorination or halogenation. Chlorination reactions of hydroxyquinolines suffer from the generation of viscous tars and decomposition products that are difficult to remove. The yields in chlorination reaction are variable and range from 24-60%.

To date there has not been any description in the art to a catalytic or milder method of preparing quinolines that does not require high temperatures. The present invention provides a novel milder synthesis that overcomes the scale-up issues of the prior art.

### SUMMARY OF THE INVENTION

The present invention provides improved methods of making such substituted 3-cyanoquinolines, intermediates obtained by the methods of the invention, and the substituted 3-cyanoquinolines made by the methods of the invention.

The methods of making the 3-cyanoquinolines utilize two separate pathways. Both pathways result in the production of N-aryl-2-propene derivatives which are then treated with phosphoryl chloride to provide 3-cyanoquinolines.

A first embodiment of this invention is directed to a method of preparing a substituted 3-cyanoquinoline which comprises the step of treating an N-aryl-2-propene represented by formula III: 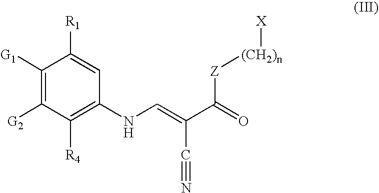
with POCl₃ to form a substituted 3-cyanoquinoline represented by formula XI: 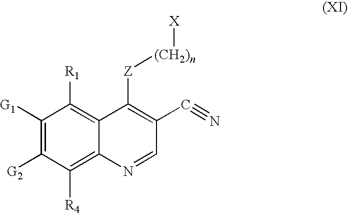
wherein:
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino; or
X is a radical having the formula: 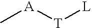
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; T is bonded to a carbon of A and is:
—NH(CH₂)ₘ—, —O(CH₂)ₘ—, —S(CH₂)ₘ—, —NR —(CH₂)ₘ—, —(CH₂)ₘ—, —(CH₂)ₘNH—, —(CH₂)ₘO—, —(CH₂)ₘS—, or —(CH₂)ₘNR—;
L is an unsubstituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂ NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z is —NH—, —O—, —S—, or —NR—;
R is alkyl of 1-6 carbon atoms, or carboalkyl of 2-7 carbon atoms;
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 14 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 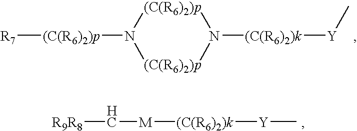
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₁ and/or G₂ are independently selected from a protected amino group and R₂—NH—;
or if any of the substituents R₁, G₁, G₂, or R₄ are located on contiguous carbon atoms then they may be taken together as the divalent radical —O—C(R₆)₂—O—; Y is a divalent radical selected from the group consisting of 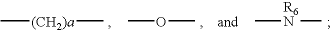
R₇ is —NR₆R₇, —OR₆, -J, —N(R₆)₃⁺, or —NR₇(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 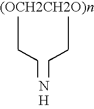
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or —(C(R₆)₂)ₛ N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of

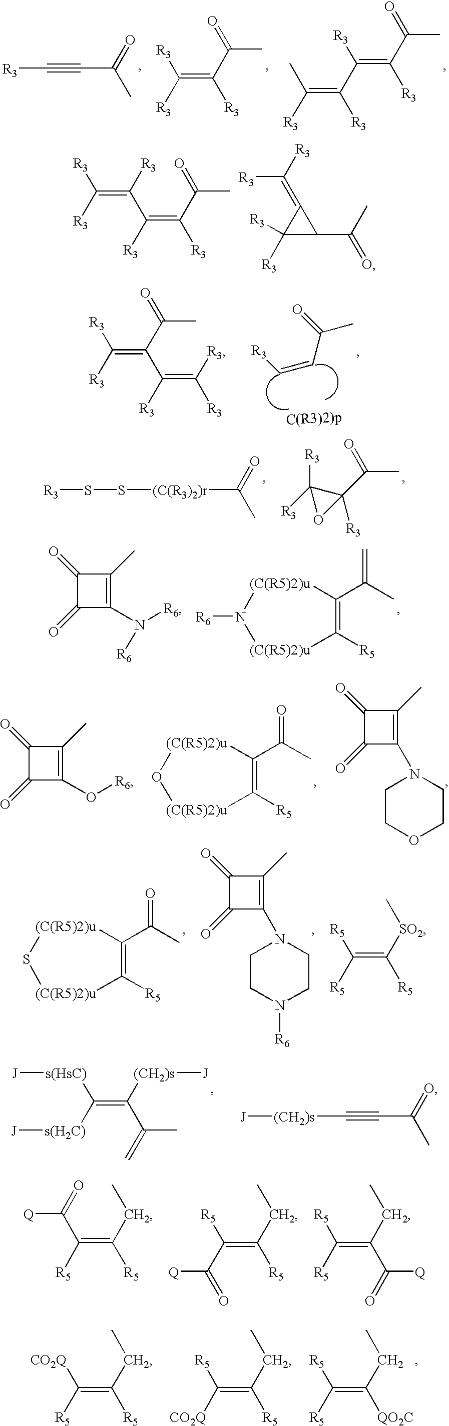

R₃ is independently hydrogen, alkyl of 1-6 carbon atoms, carboxy, carboalkoxy of 1-6 carbon atoms, phenyl, carboalkyl of 2-7 carbon atoms,

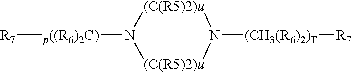

R₇—(C(R₆)₂)ₛ—, R₇—(C(R₆)₂)ₚ-M-(C(R₇)₂)ᵣ—, R₈R₉—CH-M-(C(R₆)₂)ᵣ—, or Het-(C(R₆)₂)_{q}—W—(C(R₆)₂)ᵣ—;

R₅ is independently hydrogen, alkyl of 1-6 carbon atoms, carboxy, carboalkoxy of 1-6 carbon atoms, phenyl, carboalkyl of 2-7 carbon atoms,

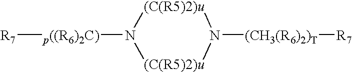

R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—; R₈, and R₉ are each, independently, —(C(R₆)₂)ᵣ NR₆R₆, or —(C(R₆)₂)ᵣOR₆;

J is independently hydrogen, chlorine, fluorine, or bromine;

Q is alkyl of 1-6 carbon atoms or hydrogen;

a=0 or 1;

g=1-6;

k=0-4;

n is 0-1;

m is 0-3;

p=2-4;

q=0-4;

r=1-4;

s=1-6;

u=0-4 and v=0-4, wherein the sum of u+v is 2-4;

or a pharmaceutically acceptable salt thereof, provided that when R₆ is alkenyl of 2-7 carbon atoms or alkynyl of 2-7 carbon atoms, such alkenyl or alkynyl moiety is bound to a nitrogen or oxygen atom through a saturated carbon atom; and provided that when Y is —NR₆— and R₇ is —NR₆R₆—N(R₆)₃⁺, or —NR₆(OR₆), then g=2-6; when M is —O— and R₇ is —OR₆ then p=1-4;

when Y is —NR₆— then k=2-4; when Y is —O— and M or W is —O— then k=1-4 when W is not a bond with Het bonded through a nitrogen atom then q=2-4 and when W is a bond with Het bonded through a nitrogen atom and Y is —O— or —NR₆— then k=2-4.

Unless stated otherwise herein, all variables set forth above, e.g. G₁, G₂, R₁, R₄, Z, X and n, apply to the formula and schemes set forth throughout this application.

In another embodiment of this invention the N-aryl-2-propene compound of formula III may be formed by condensing an N-arylformimidate of formula I 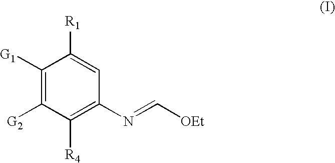
with an active methylene of formula XII

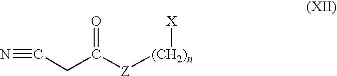

In yet another embodiment, the N-arylformimidate of formula I may be formed by reacting an arylamine of formula (XIII).

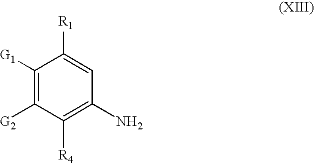

with CH(OEt)₃

In another embodiment of the invention the N-aryl-2-propene of formula III may be formed by reacting an alkoxymethylene derivative of formula II. 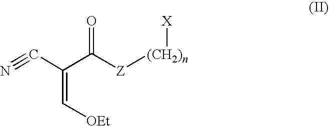
with an arylamine of formula XIII

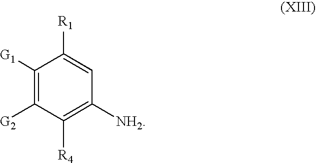

In a further embodiment of the invention, the alkoxymethylene of formula II may be formed by condensing an active methylene of formula XII 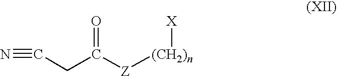
with CH(OEt)₃.

The present invention also provides for the intermediates produced by the methods of the present invention. In one embodiment of the invention, the intermediates are of formulas I, II and III below.

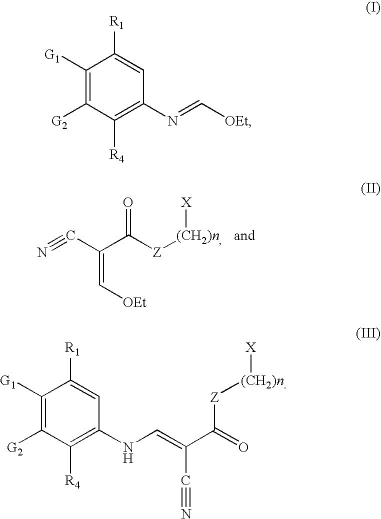

In another embodiment of the invention, the intermediates are of formulas IV and V below: 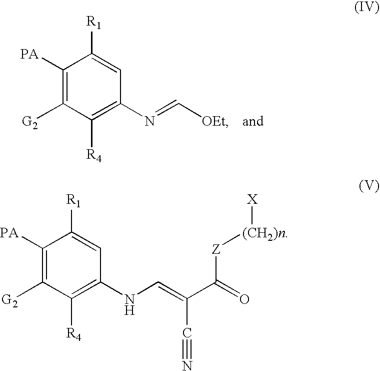
wherein PA is a protected amino group.

In a further embodiment of the invention, the intermediates are of formulas VII, VII′, VIII, IX and IX′ below:

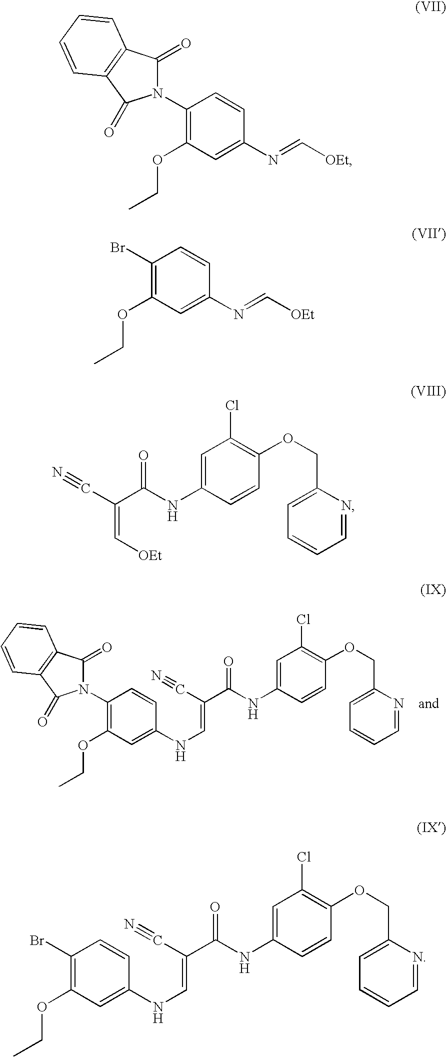

The present invention also provides for 3-cyanoquinoline derivatives produced by the methods of the present invention. Accordingly, the invention provides a 3-cyanoquinoline of formulas VI, X, X′ and XI produced by the methods of the present invention:

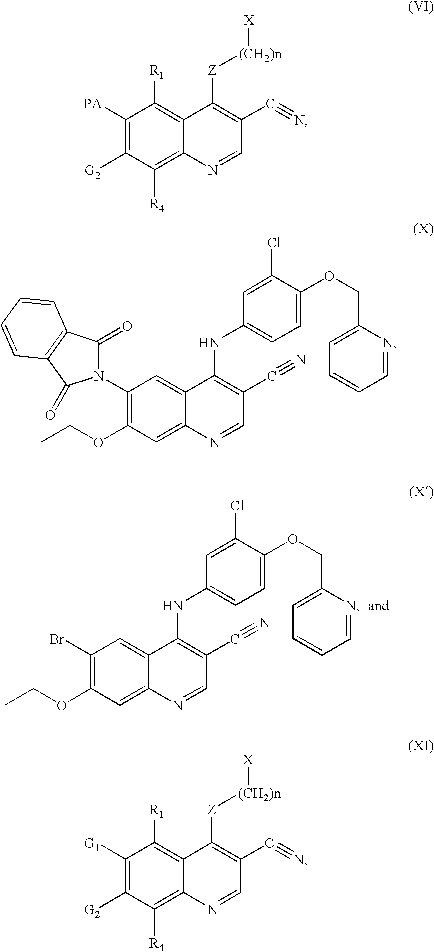

### DETAILED DESCRIPTION OF THE INVENTION

The present invention provides improved methods of making substituted 3-cyanoquinolines, intermediates obtained by the methods of the invention, and substituted 3-cyanoquinolines made by the methods of the invention.

As noted above, the methods of making the 3-cyanoquinolines utilize two separate pathways. Both pathways include the reaction of arylamines, orthoformates and active methylenes and both pathways result in the production of N-aryl-2-propene derivatives. The methods of the present invention avoid many of the hindrances of the prior synthetic pathways, since they do not involve heating to high temperatures or using microwave irradiation. Thus, the current methods can be easily adapted to large-scale preparation of 3-cyanoquinolines. Also, the chlorination step used in prior synthetic methods is avoided. This is an improvement because the chlorination of quinolines are known to suffer from the formation of viscous tars and decomposition products, which result in lower yields of the desired product and impurities that are difficult to remove. Therefore, syntheses involving chlorination of quinolines are not very practical for use in large-scale synthesis of 3-cyanoquinolines. The current method is also advantageous because it readily allows for the synthesis of many 3-cyanoquinolines derivatives with varying moieties substituted at the 4-position of the quinoline ring system.

In one embodiment of the invention, the arylamine includes a protected amino group such as phthalimide, cyclic imides, maleimide, 2,3-dichloromaleamide, succinimide, dihydrophthalimide, and 2,5-dimethylpyrrole. Arylamines which may be used in the methods of the present invention are described, for example, in U.S. Pat. No. 4,873,338 of Wiesen et al., and U.S. Pat. No. 4,617,316 of Plummer et al., all of which are incorporated herein by reference in their entirety.

In one embodiment of the invention, an arylamine is reacted with an orthoformate to produce an N-arylformimidate, which is then condensed with an active methylene to produce an N-aryl-2-propene.

In another embodiment of the invention, an active methylene compound is condensed with an orthoformate to produce an alkoxymethylene derivative which is then reacted with an arylamine to give an N-aryl-2-propene. As mentioned above, in both schemes, the N-aryl-2-propene is then treated with phosphoryl chloride to produce the 3-cyanoquinolines of the present invention. In yet another embodiment, the arylamine includes at least one protected amino group.

One embodiment of the invention is depicted in Scheme 1 below which shows both pathways of the methods of the invention.

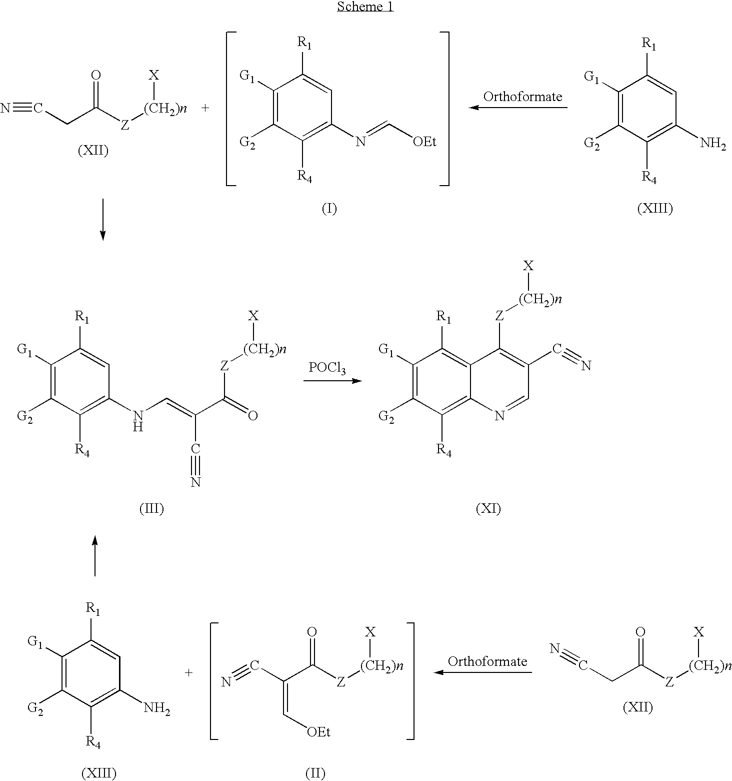

Another embodiment of the invention is depicted by Scheme 2 below which shows the embodiments of the two pathways of the methods of the present invention. 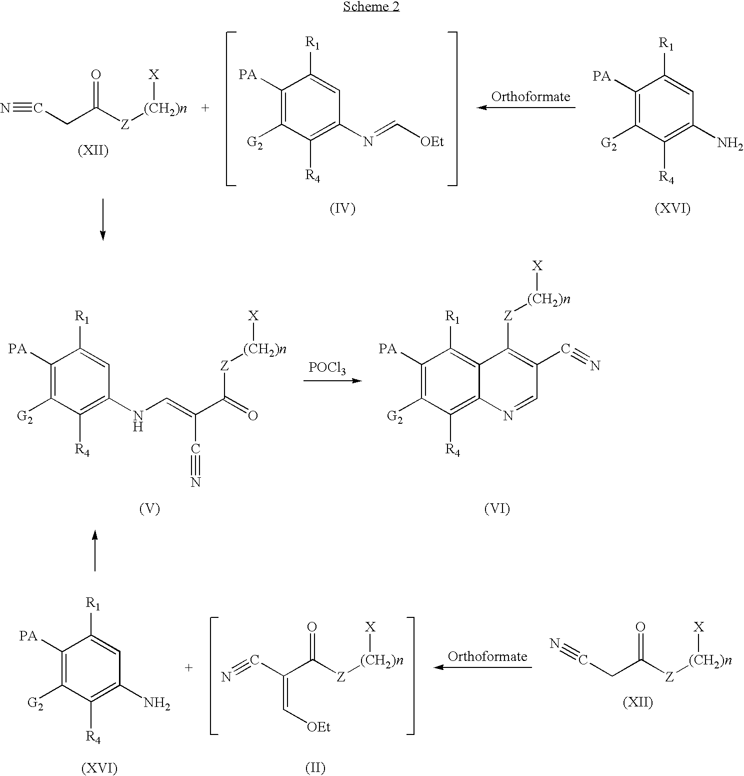
where PA represents a protected amino group.

Specific embodiments of the present invention are depicted in Schemes 3a and 3b below which show embodiments of the two pathways of the methods of the present invention. In scheme 3a, G₁ is a protected amine. Scheme 3b illustrates where G₁ is bromo. Compounds with halogens at the 6-position can be readily reacted with reagents, such as amines and alcohols, to form other derivatives encompassed within the present invention.

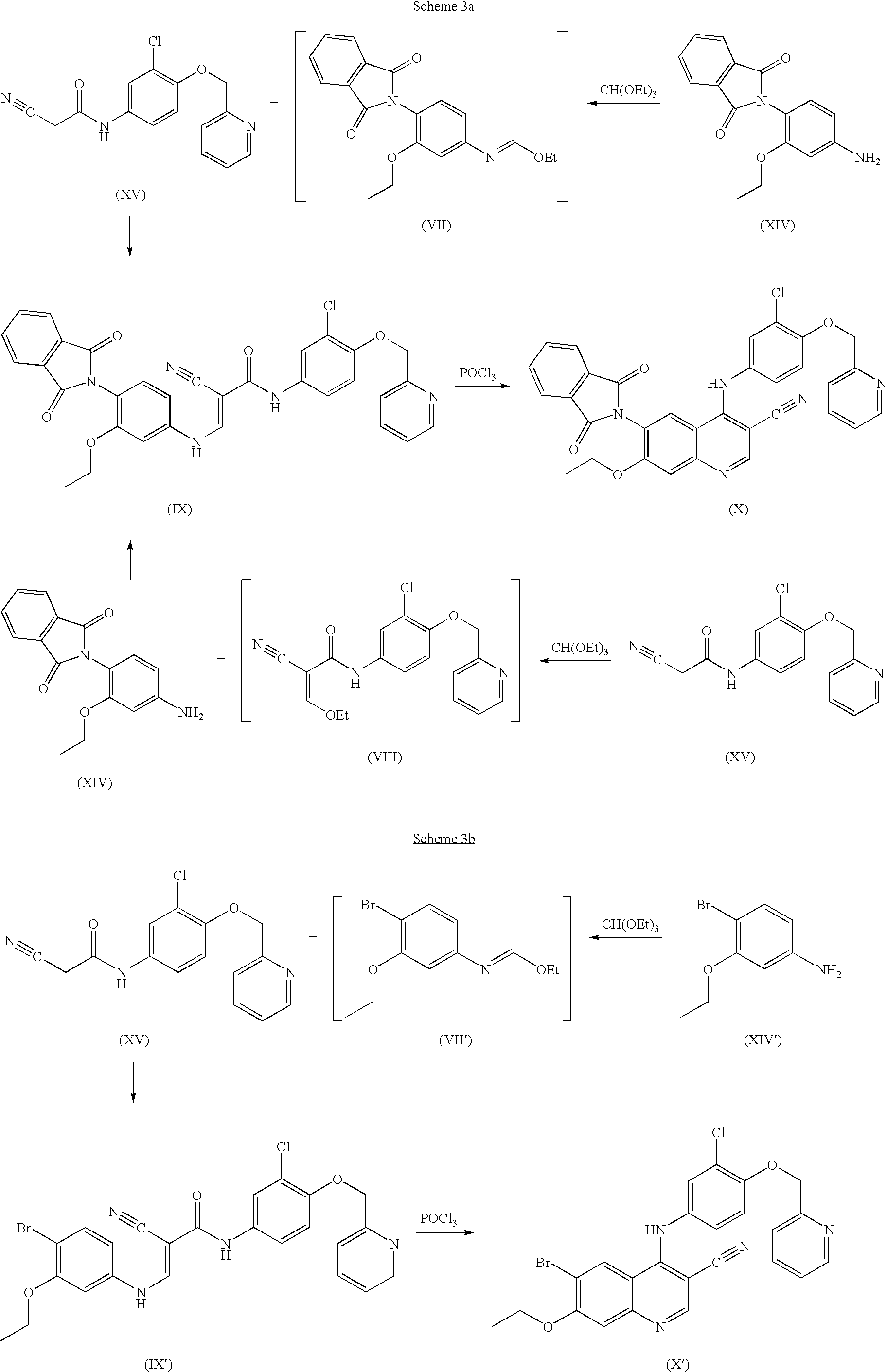

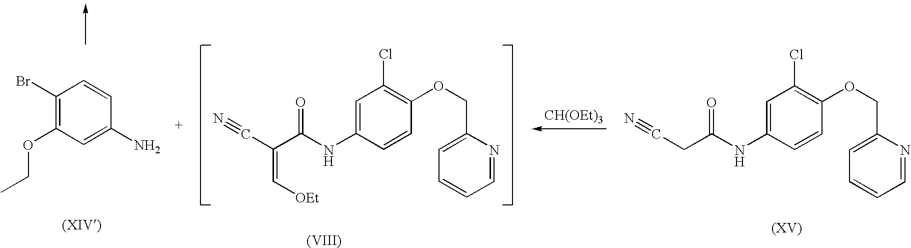

Scheme 1 includes two embodiments of the invention, which represent the two separate pathways, a first of which is depicted by Scheme 4 below. 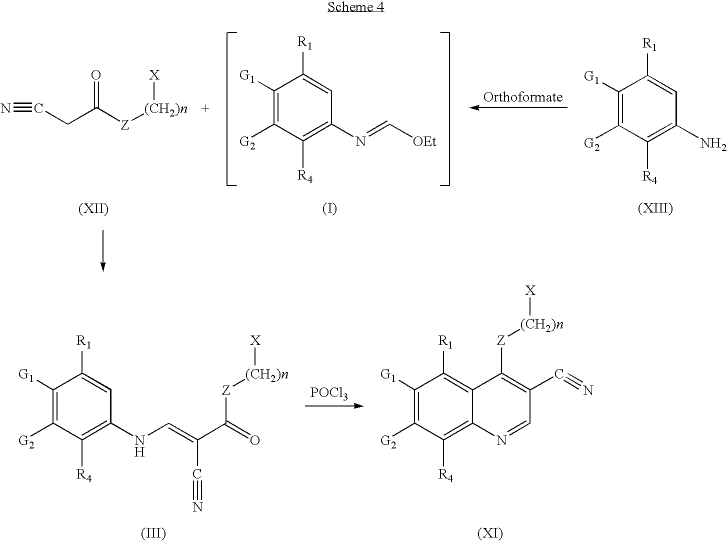
wherein X, Z, G₁, G₂, R₁ and R₄ are as described previously.

The term protected amino group (PA) refers to an amine or amino group having or forming a “protecting group” which refers to a group introduced into a molecule to protect a sensitive functional group or specific position on the molecule from reacting when the molecule is exposed to reagents or conditions to transform or react another part of the molecule. Thereafter the protecting group can be removed. Suitable protecting groups are well known in the art and include acid-labile, base-labile, photoremovable, or removable under neutral conditions. See, e.g., Green, Protecting Groups in Organic Synthesis, Wiley 1991, 2^{nd} ed., pp. 309-405, which is incorporated herein by reference. Exemplary protected amino groups include acetamides, benzamides, cyclic imides (e.g., phthalimide, maleimide, 2,3-dichloromaleimide, succinimide, dihydrophthalimide), pyrroles (e.g. 2,5-dimethylpyrrole), tert-butoxycarbonyl protected amine and benzyloxycarbonyl protected amide.

Cyclicimides are particularly useful protecting groups for masking primary amines. They are formed by reacting primary amine to be masked with a reagent such as phthalic anhydride or maleamic anhydride, thereby incorporating the amine into the cyclicimide, as shown below.

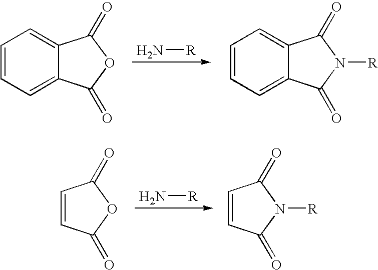

Thereafter, the cyclicimides can be cleaved under a variety of conditions, such as NH₄OH, to give the primary amine in good yield. See Green at pp. 358-359. In one embodiment of the present invention NH₄OH is used to cleave the phthalimide protecting group. This is best accomplished using multiple equivalents of NH₄OH relative to the protected compound, wherein 10 equivalents are effective, with 25 equivalents being even more efficient. 2,5-Dimethylpyrrole operates similarly.

The second embodiment of Scheme 1 is depicted in Scheme 5 below. 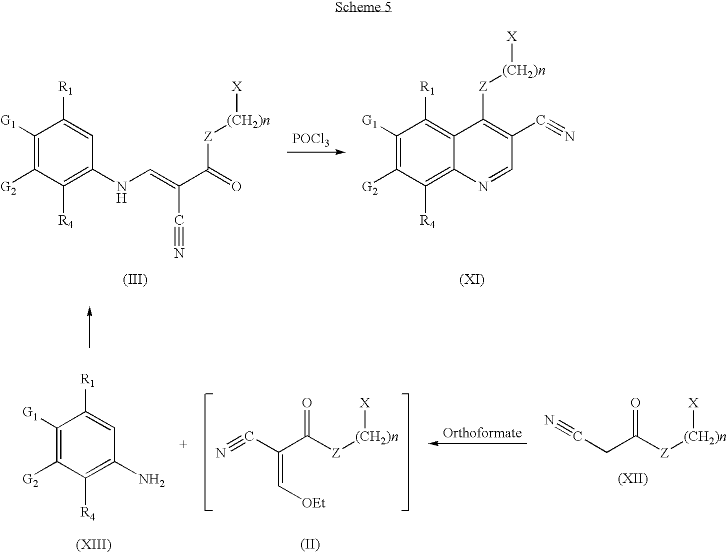
wherein X, Z, G₁, G₂, R₁ and R₄ are as described previously.

In another embodiment of Schemes 4 and 5, X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino.

In another embodiment of the invention, X is a radical having the formula: 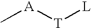
wherein A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;

T is bonded to a carbon of A and is:

—NH(CH₂)ₘ—, —O(CH₂)ₘ—, —S(CH₂)ₘ—, —NR —(CH₂)ₘ—, —(CH₂)ₘ—, —(CH₂)ₘNH—, —(CH₂)ₘO—, —(CH₂)ₘS—, or —(CH₂)ₘNR—;

L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂ NH— or —CH₂O—; or

L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino.

In a further embodiment of the methods depicted by Schemes 4 and 5, G₁ is a protected amino group (PA). In a yet another embodiment of the invention, the protected amino group is phthalimide.

The two embodiments of the pathways shown in Scheme 2 above, are depicted in Scheme 6 and 7 below. 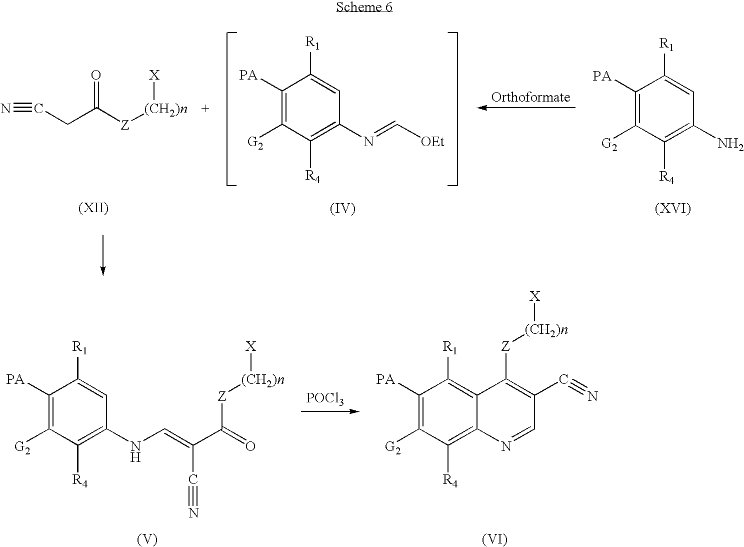
and

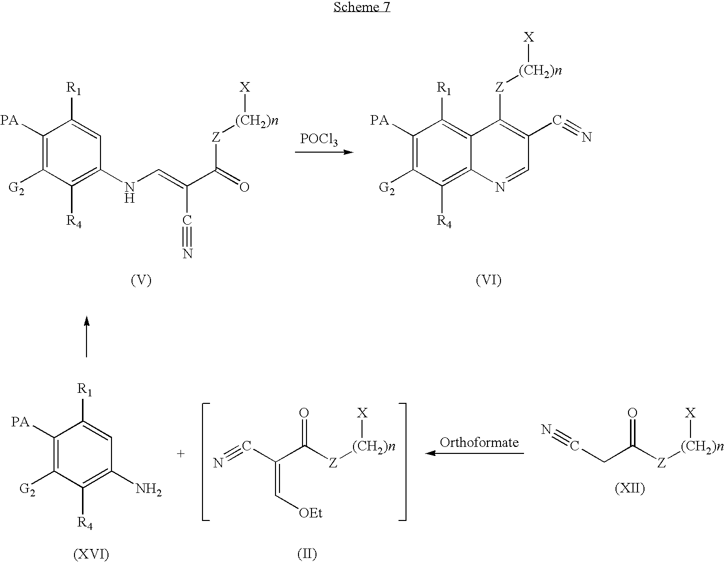

wherein X, Z, G₂, R₁ and R₄ are as described above for Schemes 4 and 5, and wherein PA us a protected amino group. In a particularly preferred embodiment of Schemes 6 and 7, PA is phthalimide.

The four embodiments depicted in Schemes 3a and 3b above are separately depicted in Schemes 8 and 9 below.

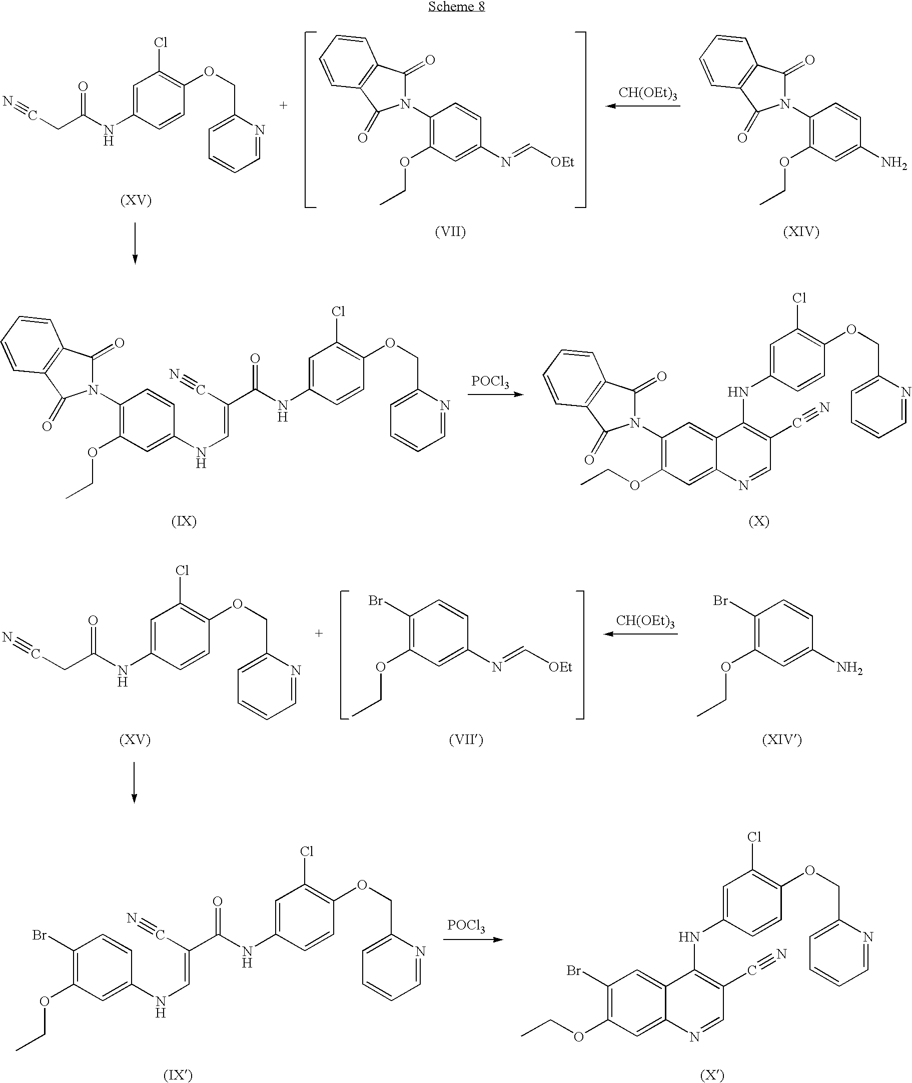

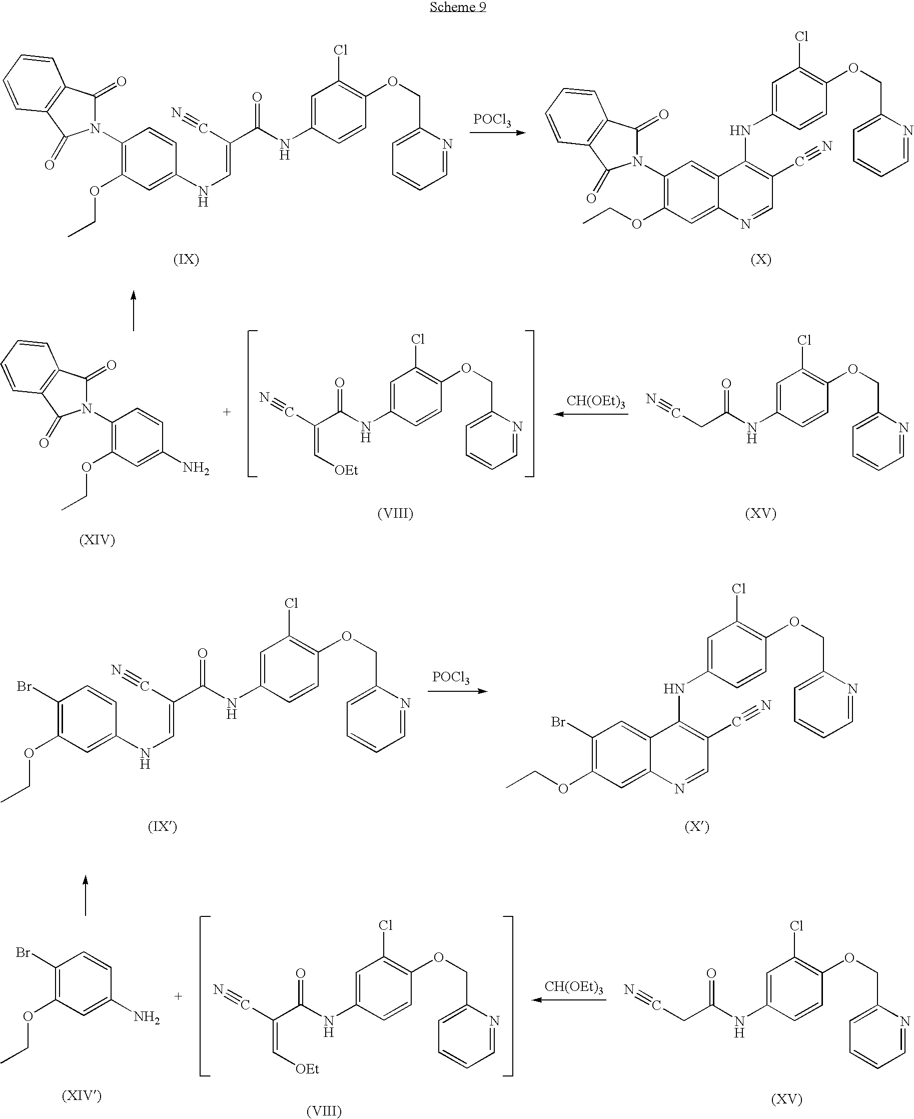

The present invention also provides for the intermediates produced by the methods of the present invention. In one embodiment of the invention, the intermediates are of formulas I, II and III below. 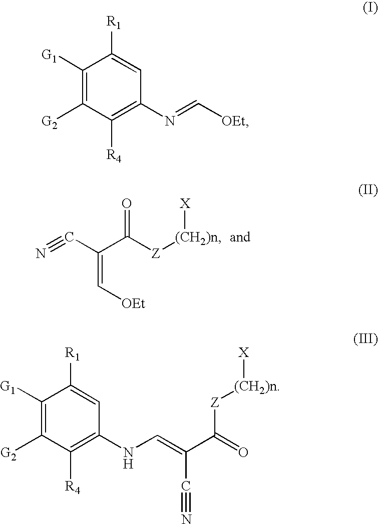
wherein X, Z, G₁, G₂, R₁ and R₄ are as previously described.

In another embodiment of the invention, the intermediates are of Formulas IV and V below: 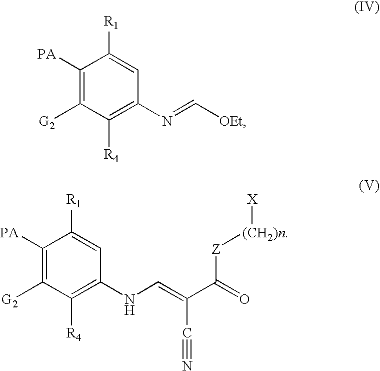
wherein X, Z, PA, G₂, R₁ and R₄ are as defined previously.

In a further embodiment of the invention, the intermediates are compounds represented by the Formulas VII, VII′, VIII, IX and IX′ below:

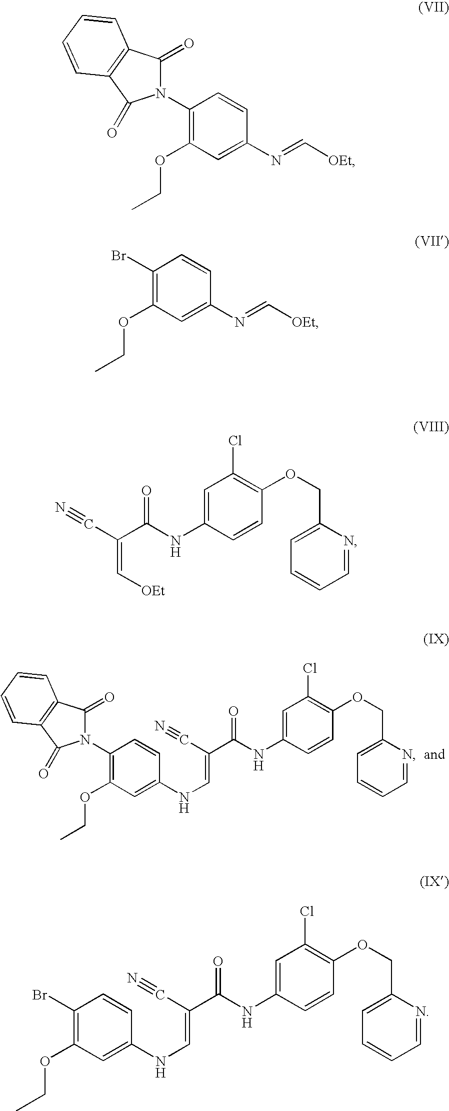

The present invention also provides for 3-cyanoquinoline derivatives produced by the methods of the present invention. Accordingly, the invention provides a 3-cyanoquinoline of Formula VI, produced by the methods depicted in Schemes 6 and 7 above, and a 3-cyanoquinoline of Formula XI produced by the methods depicted in Schemes 4 and 5 above: 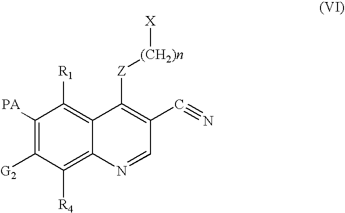
wherein X, Z, PA, G₂, R₁ and R₄ are as previously described; 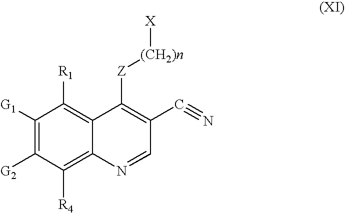
wherein X, G₁, G₂, R₁ and R₄ are as previously described.

Specific 3-cyanoquinolines of the invention are represented by the compounds of formula X and X′.

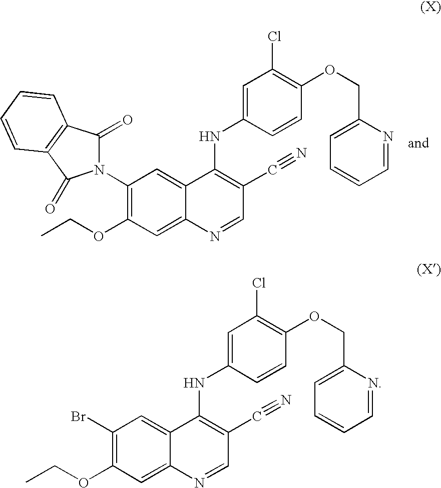

### EXAMPLES

The following examples are offered to illustrate, but not to limit the present invention.

### Example 1

### Preparation of (E/Z)-3-(4-bromo-3-ethoxyanilino)-N-[3-chloro-4-(2-pyridinyl-methoxy)phenyl]-2-cyano-2-propenamide

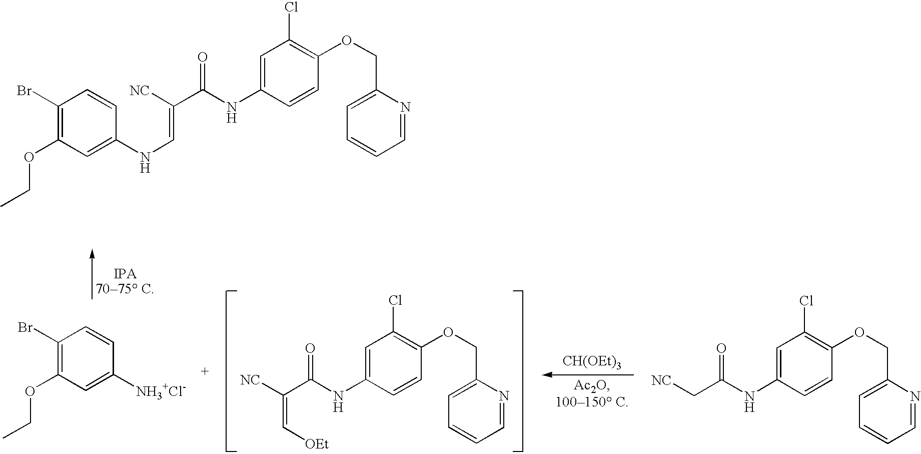

To a 3-necked 50 ml flask was charged cyanoacetamide (0.50 g, 1.7 mmole) in triethylorthoformate (2.45 g, 2.75 ml, d=0.89 g/ml). The mixture was heated to 50-60° C. and acetic anhydride (0.42 g, 0.39 ml, 4.1 mmole, 2.5 eq, d=1.08 g/ml) was added. The flask was heated to 100-105° C. and held for a minimum of 4 h and then cooled to 70-75° C. A solution of 3-ethoxy-4-bromoaniline hydrochloride (0.42 g, 1.67 mmole) in isopropanol (5 ml) was added. The mixture was stirred for 3 h and cooled to room temperature. Water was added and the mixture extracted with ethyl acetate. The organic layer was washed with brine, dried over sodium sulfate and stripped to dryness. The residue was dissolved in acetonitrile (10 ml) and water (10 ml) was added dropwise to precipitate the product. The product was filtered on a Buchner funnel to give 0.11 g of the title compound.

### Example 2

### Preparation of 3-ethoxy-4-(N-phthalimidyl)nitrobenzene

To a 5-L multi-necked flask equipped with mechanical stirrer, temperature probe, condenser and nitrogen protection was charged 2-amino-5-nitrophenol (116 g, 0.75 mole) and phthalic anhydride (223 g, 1.51 mol, 2.0 eq) followed by glacial acetic acid (1.13 L). The reaction mixture was heated to (115-120° C.) and held for 2.5-3 hr until the reaction was complete by HPLC or TLC. The mixture was cooled to room temperature and water was added (0.5 L) over 10 mins. The mixture was held for 1 h and the solids were filtered on a 15 cm diameter Buchner funnel and washed with water (2×0.5 L). The solids were transferred back to the 5-L flask, water was added (2.32 L) and the mixture stirred at room temperature for a minimum of 30 mins. The mixture was filtered and washed with water (2×0.5 L). The product was dried at 60° C. for 23 hr in a vacuum oven to give 210 g (99%) as a tan solid. ¹H NMR: δ (DMSO-d₆) 10.98 (s, 1H, OH), 8.10-7.85 (m, 4H, phthalimide), 7.82 (d, 1H, Ar), 7.80 (d, 1H, Ar), 7.63 (dd, 1H, Ar).

To a 5-L multi-necked flask equipped with mechanical stirrer, temperature probe, condenser and nitrogen protection was charged N-(2-hydroxy-4-nitrophenyl)phthalimide (208 g, 0.73 mol) and DMF (1.04 L). The mixture was stirred at room temperature until a solution was obtained. Potassium carbonate (0.15 kg, 1.5 eq.) was added in portions until the pH of the mixture was 9. The suspension was heated to 60-65° C. and ethyl bromide (88 g, 0.80 mol, 1.1 eq.) was added dropwise over 20 min. After complete addition, the mixture was held for a minimum of 30 mins and then cooled to room temperature. Water (2.08 L) was added over 30 mins while maintaining the pot temperature at 20-25° C. and the mixture was held for an additional 1 hr. The mixture was filtered on a 15 cm diameter Buchner funnel and washed with warm (45-50° C.) water (3×0.2 L). The wet cake was transferred back to the 5-L flask, water (2.08 L) was added and the mixture slurried for a minimum of 30 mins at 45-50° C. The mixture was filtered and washed with water (0.2 L). The product was dried at 60° C. for 20 hr in a vacuum oven to give the titled compound (154 g, 67%). ¹H NMR: δ (DMSO-d₆) 8.10-7.92 (m, 6H, Ar), 7.73 (d, 1H, Ar), 4.23 (q, 2H, CH₃CH₂O), 1.21 (t, 3H, CH₃CH₂O).

### Example 3

### Preparation of N-[3-Chloro-4-(2-pyridinylmethoxy)]phenyl-2-cyanoacetamide

In a 12-L multi-necked flask, 2-pyridyl carbinol (0.13 kg, 1.19 mole, 1.05 eq) was dissolved in acetonitrile (0.88 L) and to it was added potassium hydroxide flakes (85%) (80 g, 1.25 eq). The resulting suspension was warmed to 35° C. A solution of 3-chloro-4-fluoronitrobenzene (0.20 kg, 1.14 mol) in acetonitrile (1.0 L) was added at 35-40° C. The mixture was held for 18 h until reaction completion. The mixture was then cooled back to 20-25° C., quenched with water (4 L) and the resulting slurry was filtered and washed with water (3×200 mL). The resulting product was isolated as a tan solid (251 g, 84% yield).

A mixture of 3-chloro-4-(2-pyridylmethoxy)nitrobenzene (0.149 kg, 0.56 mole) and 2% (w/w) of 5% Pt/C (6.0 g, 50% water wet) in tetrahydrofuran (0.895 L) was hydrogenated in a 2-L stainless steel Parr reactor at 25 psi, 25° C. for a minimum of 8 h. The mixture was filtered through a celite pad (50 g, 15 cm diameter) and washed with tetrahydrofuran (0.45 L). The filtrate was distilled to a volume of 0.30 L and the concentrate was transferred to a 2-L multi-neck flask and used as is in the next step.

To the 2-L flask equipped with mechanical stirrer, temperature probe, claisen head and condenser was added ethylcyanoacetate (0.421 kg, 3.72 mole, 6.6 eq.). The reaction mixture was heated to (100-115° C.) while removing tetrahydrofuran and ethanol. The temperature was raised to ˜125° C. and the mixture was held for a minimum of 24 h until the aniline starting material was consumed and no distillate was collected. The mixture was cooled to room temperature over 1 h. At 50-60° C., solids crystallized out and ethyl acetate (0.15 L) was added. The mixture was further cooled to 0-10° C. and held for 1 h. The mixture was filtered on a 15 cm diameter Buchner funnel and washed with 50 mL of the filtrate followed by pre-cooled (0-10° C.) ethyl acetate (0.15 L). The product was dried at 60° C. for a minimum of 16 h in a vacuum oven to give the titled compound (0.12 kg, 71%) as a brown solid. The product was purified by slurrying in cold ethyl acetate (1-1.3 volumes) for 1 hr. ¹H NMR: δ (DMSO-d₆) 10.31 (s, 1H, NH), 8.58 (dd, 1H, Ar), 7.86 (dt, 1H, Ar), 7.75 (d, 1H, Ar), 7.55 (d, 1H, Ar), 7.39-7.32 (m, 2H, Ar), 7.21 (d, 1H, Ar), 5.25 (s, 2H, OCH₂Pyr), 3.88 (s, 2H, NCCH₂CO).

### Example 4

### Preparation of (E/Z) 3-[3-ethoxy-4-(N-phthalimidyl)]anilino-N-[3-chloro-4-(2-pyridinylmethoxy)]phenyl-2-cyano-2-propenamide

A mixture of 3-ethoxy-4-(N-phthalimidyl)nitrobenzene (0.135 kg, 0.43 mole) and 4% (w/w) of 10% Pd/C (5.42 g, 50% water wet) in tetrahydrofuran (1.08 L) was hydrogenated in a 2-L stainless steel Parr reactor at 70 psi, 50° C. for a minimum of 10 h. The mixture was filtered through a celite pad (100 g, 15 cm diameter) into a 1-neck 3-L flask and washed with tetrahydrofuran (3×0.14 L). The filtrate was distilled to a volume of 0.55 L and to the concentrate was added n-propanol (0.75 L). The mixture was distilled to a volume of −0.41 L and the concentrate was used as is in the next step. Taking an aliquot and concentrating to dryness obtained an analytical reference sample.

The concentrate was transferred to a 3-L multi-necked flask equipped with an agitator, temperature probe, condenser and nitrogen protection. The flask was charged with n-propanol (0.91 L) followed by N-[3-Chloro-4-(2-pyridinylmethoxy)]phenyl-2-cyanoacetamide (19 g, 0.39 mole, 0.91 eq.). The mixture was heated to 75-80° C. The first of three portions of triethylorthoformate (64.3 g, 72 ml, 0.43 mole, d=0.89 g/ml) was added and the mixture heated to reflux (95-100° C.). After 2.5 h, the second portion of triethylorthoformate was added (64.3 g, 72 ml, 0.43 mole). After 19.5 hr, the third portion of triethylorthoformate was added (64.3 g, 72 ml, 0.43 mole). The third portion may be added 2.5 hr after the second portion. The mixture was held for a minimum of 6.5 hr at 95° C. (total of 28.5 h). The mixture was cooled to 0-10° C. and held for 1 hr. The mixture was filtered on an 18 cm diameter Buchner funnel and washed with filtrate (150 ml) followed by chilled (0-10° C.) n-propanol (4×0.15 L). The weight of the wet cake was 252 g. (estimated LOD of −12%).

The wet cake was purified from acetonitrile. The wet cake (186 g) was transferred to a 5-L multi-necked flask equipped with mechanical stirrer, condenser, temperature probe and nitrogen protection. The flask was charged with acetonitrile (2.42 L), heated to 65-70° C. and held for a minimum of 30 min. The mixture was cooled to 60° C., filtered on a 15 cm diameter Buchner funnel and washed with acetonitrile (3×0.18 L). The product was dried at 60° C., full vacuum for 18 h to give 3-[3-ethoxy-4-(N-phthalimidyl)]anilino-N-[3-chloro-4-(2-pyridinylmethoxy)]phenyl-2-cyano-2-propenamide (122 g, 75% recovery based on dry weight) as a beige solid. Overall yield over 2 steps: 53%. ¹H NMR (aniline): δ (DMSO-d₆) 7.96-7.86 (m, 4H, phthalimide), 6.90 (d, 1H, Ar), 6.34 (d, 1H, Ar), 6.22 (dd, 1H, Ar), 5.37 (d, 2H, NH₂), 3.90 (q, 2H, CH₃CH₂O), 1.11 (t, 3H, CH₃CH₂O). ¹H NMR (propenamide): δ (DMSO-d₆) 11.45 (d, 1H, NHCH═C), 9.58 (d, 1H, CONH), 8.59 (m, 1H, Ar), 8.54 (d, 1H, NHCH═C), 8.00-7.84 (m, 6H, Ar), 7.57 (d, 1H, Ar), 7.52 (dd, 1H, Ar), 7.39-7.34 (m, 3H, Ar), 7.21 (d, 1H, Ar), 7.11 (dd, 1H, Ar), 5.27 (s, 2H, OCH₂Pyr), 4.11 (q, 2H, OCH₂CH₃), 1.16 (t, 3H, OCH₂CH₃).

### Example 5

### Preparation of 3-cyano-4-[3-chloro-4-(2-pyridinylmethoxy)]anilino-7-ethoxy-N-phthalimidylquinoline

To a 5-L multi-necked flask equipped with an agitator, temperature probe, condenser and nitrogen protection was charged 3-[3-ethoxy-4-(N-phthalimidyl)]anilino-N-[3-chloro-4-(2-pyridinylmethoxy)]phenyl-2-cyano-2-propenamide (0.12 kg, 0.20 mole) and suspended in acetonitrile (1.20 L) and methanol (0.06 L). A sodium hydroxide (40 g in 1 L water) scrubber system was set up. The mixture was heated to 60-65° C. and phosphorus oxychloride (0.31 kg, 0.19 L, 10.0 eq, d=1.645 g/ml) was added dropwise keeping the pot temperature at 60-70° C. The mixture eventually became thinner and deep red (after ˜6 hr). The mixture was held for a minimum of 18 h and then cooled to 0-10° C. Water (0.60 L) was added keeping the pot temperature <20° C. The pH of the mixture was adjusted to 8-10 using 28% ammonium hydroxide (−0.95 L). The 2-phase mixture was filtered on a 15 cm diameter Buchner funnel and washed with 2:1 acetonitrile:water (2×0.12 L). The wet cake was transferred to the 3-L flask, water (1.20 L) was added and the mixture slurried for a minimum of 30 mins at 45-50° C. The mixture was filtered at 45° C. on a 15 cm diameter Buchner funnel and washed with water (3×0.20 L) until the pH of the final wash was 7-8. The solid was dried at 60° C., in a vacuum oven for 24 hr to give the desired compound (88 g, 76%) as an orange solid. ¹H NMR: δ (DMSO-d₆) 9.72 (s, 1H, Ar), 8.58 (m, 2H, Ar), 8.53 (s, 1H, Ar), 8.08-7.96 (m, 4H, phthalimide), 7.87 (m, 1H, Ar), 7.55 (m, 3H, Ar), 7.37 (m, 1H, Ar), 7.28 (s, 1H, Ar), 5.29 (s, 2H, OCH₂Pyr), 4.24 (q, 2H, OCH₂CH₃), 1.22 (t, 3H, OCH₂CH₃).

### Example 6

### Preparation of 6-amino-4-[3-chloro-4-(2-pyridinylmethoxy)]anilino-3-cyano-7-ethoxyquinoline

To a 3-L multi-necked flask equipped with an agitator, temperature probe, condenser and nitrogen protection was charged 3-cyano-4-[3-chloro-4-(2-pyridinylmethoxy)]anilino-7-ethoxy-N-phthalimidylquinoline (0.085 kg, 0.147 mole) and suspended in ethanol (ASDQ #2, 0.68 L). Next, 28% ammonium hydroxide (0.5 L of a 7.4 M solution, 25 eq.) was added. The suspension was heated to 62-68° C. and held for a minimum of 2 hr. The reaction was cooled to room temperature, filtered on a 15 cm diameter Buchner funnel, and washed with ethanol (2×85 ml). The solid was dried at 65° C., in a vacuum oven for 20 hr to give the titled compound (61 g, 92% uncorrected for strength) as an orange solid. ¹H NMR: δ (DMSO-d₆) 9.08 (s, 1H, NH), 8.59 (d, 1H, Ar), 8.31 (s, 1H, Ar), 7.90-7.84 (m, 1H, Ar), 7.58 (s, 1H, Ar), 7.40-7.34 (m, 1H, Ar), 7.26-7.19 (m, 4H, Ar), 7.09-7.05 (m, 1H, Ar), 5.40 (d, 2H, NH₂), 5.25 (s, 2H, OCH₂Pyr), 4.23 (q, 2H, OCH₂CH₃), 1.45 (t, 3H, OCH₂CH₃).

### Example 7

### Preparation of (E)-N-{4-[3-chloro-4-(2-pyridinylmethoxy)anilino]-3-cyano-7-ethoxy-6-quinolinyl}-4-(dimethylamino)-2-butenamide free base.

A solution of 4-N,N-dimethylaminocrotonic acid hydrochloride (22.3 g, 0.135 mol, 2.0 eq.) in tetrahydrofuran (0.225 L) and a catalytic amount of dimethylformamide (0.5 mL) was cooled to 0-5° C. Oxalyl chloride (11.4 mL, 0.131 mol, 1.95 eq) was added dropwise over 15 min. The mixture was then warmed to 25-30° C. and stirred for 2 h then cooled to 0-5° C. N-Methyl-2-pyrrolidinone (15 ml) was added. A filtered solution of warm (30° C.) 6-amino-4-[3-chloro-4-(2-pyridylmethoxy)]anilino-3-cyano-7-ethoxy-quinoline (30 g, 0.067 mole, 1.0 eq.) in N-methyl-2-pyrrolidinone (0.27 L) was added dropwise over 30 min keeping the temperature 0-10° C. The mixture was stirred for a minimum of 20 h. Upon completion, the reaction was quenched with water (0.36 L), held for 30 min and then warmed to 40-45° C. Aqueous sodium hydroxide (19 g in 0.15 L water) was added over 30 min to bring the pH to 9-10 followed by adding water (0.39 L). The mixture was stirred for 1 hr, then cooled to room temperature. The resulting precipitates were filtered and washed with water (3×60 mL) until the pH of the washes were 7-8. The wet solids were heated to reflux (70-75° C.) in 1.5:1 acetonitrile:tetrahydrofuran (0.33 L) and the solution cooled over 2 h to room temperature. The product was filtered and washed with cold 1.5:1 acetonitrile:tetrahydrofuran (3×0.01 L). The product was dried (60° C., 10 mm Hg, 24 h) to give the titled compound (19.4 g, 52% uncorrected for strength). ¹H NMR: δ (DMSO-d₆) 9.59 (s, 1H, NH), 9.47 (s, 1H, NH), 8.96 (s, 1H, Ar), 8.60 (dd, 1H, Ar), 8.47 (s, 1H, Ar), 7.87 (dd, 1H, Ar), 7.58 (d, 1H, Ar), 7.39-7.34 (m, 3H, Ar), 7.27-7.20 (m, 2H, Ar), 6.81-6.73 (m, 1H, CH₂—CH═CH—), 6.59 (d, 1H, CH₂—CH═CH—), 5.28 (s, 2H, OCH₂Pyr), 4.31 (q, 2H, OCH₂CH₃), 3.09 (d, 2H, N—(CH₂), 2.18 (s, 6H, N(CH₃)₂), 1.47 (t, 3H, OCH₂CH₃).

### Example 8

### Preparation of (E)-N-{4-[3-chloro-4-(2-pyridinylmethoxy)anilino]-3-cyano-7-ethoxy-6-quinolinyl}-4-(dimethylamino)-2-butenamide maleate

(E)-N-{4-[3-chloro-4-(2-pyridinylmethoxy)anilino]-3-cyano-7-ethoxy-6-quinolinyl}-4-di-methylamino)-2-butenamide crude free base (17 g, 0.027 mole, 88% strength) and maleic acid (3.60 g, 0.031 mole) were dissolved at 50-60° C. in a 5% water/n-propanol mixture (0.12 L) and stirred for 15 min. To the hot solution was added charcoal (1.7 g) and the mixture stirred for 20 min. The hot solution was clarified and cooled to room temperature and held for 12-15 hr. The product was filtered and washed with 5% water/n-propanol (3×0.017 L). The product was dried (60° C., 10 mm Hg, 24 h) to give the titled compound (9.83 g, 54%, uncorrected for strength). The product (7.0 g) was recrystallized from 7.5% water/propanol to give 5.7 g. DSC: 196° C. (single crystal form). ¹H NMR: δ (DMSO-d₆) 9.74 (s, 1H, NH), 9.63 (s, 1H, NH), 8.94 (s, 1H, Ar), 8.60 (dd, 1H, Ar), 8.50 (s, 1H, Ar), 7.88 (dd, 1H, Ar), 7.59 (d, 1H, Ar), 7.42-7.35 (m, 3H, Ar), 7.28-7.19 (m, 2H, Ar), 6.76 (d, 2H, —CH═CH—), 6.05 (s, 2H, HOOC—CH═CH—COOH), 5.29 (s, 2H, OCH₂Pyr), 4.33 (q, 2H, OCH₂CH₃), 3.91 (d, 2H, N—(CH₂), 2.77 (s, 6H, N(CH₃)₂), 1.45 (t, 3H, OCH₂CH₃). ¹³C NMR: δ (DMSO-d₆) 167.3, 162.4, 156.1, 153.4, 152.5, 151.2, 150.5, 149.1, 147.7, 137.0, 135.8, 134.0, 132.6, 131.6, 127.3, 125.9, 124.2, 123.0, 121.5, 121.4, 117.1, 115.6, 114.3, 113.2, 108.7, 87.3, 71.3, 64.6, 57.0, 42.3, 14.2.

### Example 9

### Preparation of N-[3-Chloro-4-(3-fluorobenzyloxy)]phenyl-2-cyanoacetamide

3-Fluorobenzyl alcohol (0.30 kg, 2.39 mole, 1.05 eq) was dissolved in acetonitrile (6.0 L) and to it was added potassium hydroxide flakes (85%) (0.16 kg, 1.25 eq). The resulting suspension was warmed to 35° C. A solution of the 3-chloro-4-fluoronitrobenzene (0.40 kg, 2.28 mol) in acetonitrile (2.0 L) was added at 35-40° C. The mixture was held for 18 h. The mixture was then cooled to ambient temperature, quenched with water (8 L) and the resulting slurry was filtered and washed with water (2×0.40 L). The resulting product was dried (45° C., 10 mmHg, 25 h) to give 0.59 kg (92% yield).

A mixture of 3-chloro-4-(3-fluorobenzyloxy)nitrobenzene (20 g, 0.071 mole) and ethanol (195 ml) was added zinc (23.2 g, 5.0 eq.). The mixture was heated to 55-60° C. A solution of ammonium chloride (7.6 g) in water (40 ml) was added over 20 min keeping the pot temperature 55-65° C. (small exotherm). The mixture was stirred for 2 h, filtered over a celite pad and washed with ethanol (2×20 ml). The filtrate was distilled to essentially dryness and 2-methyltetrahydrofuran (100 ml) was added to dissolve the product. Water (40 ml) and brine (5.2 g in 15 ml water) were added, mixed and the layers were separated. The organic layer was washed with water (30 ml) and then transferred to a 500 ml multi-necked flask.

To the 500 ml flask equipped with mechanical stirrer, temperature probe, claisen head and condenser was added ethylcyanoacetate (53.2 g, 0.47 mole, 6.6 eq.). The reaction mixture was heated to (120-125° C.) while removing 2-methyltetrahydrofuran and residual ethanol atmospherically. The mixture was held for a minimum of 24 h until the aniline starting material was consumed and no distillate was collected. The mixture was cooled to ambient temperature and isopropyl acetate (75 ml) and heptane (75 ml) were added. The mixture was mixed for 2 hr. The mixture was filtered on a 5.5 cm diameter Buchner funnel and washed with heptane. The product was dried at 45° C. overnight in a vacuum oven to give the titled compound (6.45 g, 29%) as a greyish solid (WC26280-77). ¹H NMR: δ (DMSO-d₆) 10.31 (s, 1H, NH), 7.73 (d, 1H, Ar), 7.46-7.19 (m, 6H, Ar), 5.20 (s, 2H, OCH₂Ph), 3.87 (s, 2H, NCCH₂CO).

### Example 10

### Preparation of (E/Z) 3-[3-ethoxy-4-(N-phthalimidyl)]anilino-N-[3-chloro-4-(3-fluorobenzyloxy)]phenyl-2-cyano-2-propenamide

A mixture of 3-ethoxy-4-(N-phthalimidyl)nitrobenzene (9.99 g, 0.032 mole, 1.1 eq.) and 4% (w/w) of 10% Pd/C (0.40 g, 50% water wet) in tetrahydrofuran (80 ml) was hydrogenated in a 0.2 L stainless steel Parr reactor at 70 psi, 50° C. for a minimum of 10 h. The mixture was filtered through a celite pad into a 1-neck 0.5 L flask and washed with tetrahydrofuran (3×10 ml). The filtrate was distilled to a volume of 40 ml and to the concentrate was added n-propanol (60 ml). The mixture was distilled to a volume of 40 ml and the concentrate was used as is in the next step.

The concentrate was transferred to a 0.5 L multi-necked flask equipped with an agitator, temperature probe, condenser and nitrogen protection. The flask was charged with n-propanol (60 ml) followed by N-[3-Chloro-4-(3-fluorobenzyloxy)]phenyl-2-cyanoacetamide (9.0 g, 0.029 mole, 1.0 eq.). The mixture was heated to 75-80° C. The first of three portions of triethylorthoformate (4.4 g, 4.7 ml, 0.029 mole, d=0.89 g/ml) was added and the mixture heated to reflux (95-100° C.). After 2 hr, the second portion of triethylorthoformate was added (4.4 g, 4.7 ml, 0.029 mole). After another 2 hr, the third portion of triethylorthoformate was added (4.4 g, 4.7 ml, 0.029 mole). The mixture was held for a minimum of 20 hr at 95° C. The mixture was cooled to 0-10° C. and held for 1 hr. The mixture was filtered on a Buchner funnel and washed with filtrate (10 ml) followed by chilled (0-10° C.) n-propanol (3×10 ml). The weight of the wet cake was 25.6 g. The product was dried at 60° C., full vacuum for 20 h to give the titled compound (15.7 g, 88% overall yield over 2 steps). ¹H NMR: δ (DMSO-d₆) 11.46 (d, 1H, NHCH═C), 9.58 (d, 1H, CONH), 8.54 (d, 1H, NHCH═C), 7.95-7.86 (m, 5H, Ar), 7.55-7.10 (m, 9H, Ar), 5.22 (s, 2H, OCH₂Ph), 4.10 (q, 2H, OCH₂CH₃), 1.16 (t, 3H, OCH₂CH₃).

### Example 11

### Preparation of 3-cyano-4-[3-chloro-4-(3-fluorobenzyloxy)]anilino-7-ethoxy-N-phthalimidylquinoline

To a 1-L multi-necked flask equipped with an agitator, temperature probe, condenser and nitrogen protection was charged 3-[3-ethoxy-4-(N-phthalimidyl)]anilino-N-[3-chloro-4-(3-fluorobenzyloxy)]phenyl-2-cyano-2-propenamide (15 g, 0.0245 mole) and suspended in acetonitrile (150 ml) and methanol (3.0 ml). A sodium hydroxide (40 g in 1 L water) scrubber system was set up. The mixture was heated to 65-70° C. and phosphorus oxychloride (37.6 g, 22.8 ml, 10.0 eq, d=1.645 g/ml) was added dropwise keeping the pot temperature at 70-75° C. The mixture eventually became thinner. The mixture was held for a minimum of 20 h and then cooled to 0-10° C. Water (75 ml) was added keeping the pot temperature <20° C. The pH of the mixture was adjusted to 8-10 using 28% ammonium hydroxide (˜115 ml) keeping the pot temperature 10-15° C. The 2-phase mixture was filtered on a Buchner funnel and washed with warm water (120 ml). The solid was dried at 60° C., in a vacuum oven for 25 hr to give the titled compound (11.29 g, 78% uncorrected for strength) as an orange solid. ¹H NMR: δ (DMSO-d₆) 8.59 (s, 1H, Ar), 8.53 (s, 1H, Ar), 8.07-7.96 (m, 4H, phthalimide), 7.53-7.42 (m, 3H, Ar), 7.34-7.16 (m, 5H, Ar), 5.26 (s, 2H, OCH₂Ph), 4.24 (q, 2H, OCH₂CH₃), 1.22 (t, 3H, OCH₂CH₃).

### Example 12

### Preparation of 6-amino-4-[3-chloro-4-(3-fluorobenzyloxy)]anilino-3-cyano-7-ethoxyquinoline

To a 250 ml multi-necked flask equipped with an agitator, temperature probe, condenser and nitrogen was charged 3-cyano-4-[3-chloro-4-(3-fluorobenzyloxy)]anilino-7-ethoxy-N-phthalimidylquinoline (10 g, 0.0169 mole) and suspended in ethanol (80 ml). Next, 28% ammonium hydroxide (22.8 ml of a 7.4 M solution, 25 eq.) was added. The suspension was heated to 65-70° C. and held for a minimum of 2 hr. The reaction was cooled to room temperature, filtered on a Buchner funnel, and washed with ethanol (2×10 ml) and heptanes (2×10 ml). The solid was dried at 60° C., in a vacuum oven for 24 hr to give the titled compound (5.78 g, 74%) as an orange-red solid. ¹H NMR: δ (DMSO-d₆) 9.10 (s, 1H, NH), 8.31 (s, 1H, Ar), 7.51-7.05 (m, 9H, Ar), 5.36 (s, 2H, NH₂), 5.22 (s, 2H, OCH₂Ph), 4.23 (q, 2H, OCH₂CH₃), 1.45 (t, 3H, OCH₂CH₃).

### Example 13

### Preparation of N-(3-Chloro-4-fluoro)phenyl-2-cyanoacetamide

3-Chloro-4-fluoroaniline (1.00 kg, 6.87 mole) and cyanoacetic acid (0.602 kg, 7.08 mole) were reacted together in the presence of 1,3-diisopropylcarbodiimide (0.893 kg, 1.108 L, 7.08 mole) in refluxing THF (2.67 kg, 3.0 L), similar to that reported by R. Westwood et. al. [J. Med. Chem., 39, 4608, (1996)]. The urea byproduct precipitated from the THF solution and was removed after chilling to 13±2° C. and filtration. The precipitates were washed with THF (3×1.0 L). The filtrate was then added slowly to a large volume of water (17 L) to precipitate the product. The resulting slurry of crystals was filtered, washed with water (2×0.50 L) and dried under vacuum at 45° C. for a minimum of 24 hrs to give the desire compound as an off white solid (1.25 kg, 86%). ¹H NMR: δ (DMSO-d₆) 10.48 (s, 1H, NH), 7.83 (dd, 1H, Ar), 7.45-7.32 (m, 2H, Ar), 3.90 (s, 2H, NCCH₂CO).

### Example 14

### Preparation of (E/Z) 3-[3-ethoxy-4-(N-phthalimidyl)]anilino-N-(3-chloro-4-fluoro)phenyl-2-cyano-2-propenamide

A mixture of 3-ethoxy-4-(N-phthalimidyl)nitrobenzene (20.0 g, 0.064 mole, 1.1 eq.) and 4% (w/w) of 10% Pd/C (0.80 g, 50% water wet) in tetrahydrofuran (160 ml) was hydrogenated in a 0.2 L stainless steel Parr reactor at 70 psi, 50° C. for a minimum of 10 h. The mixture was filtered through a celite pad into a 1-neck 0.5 L flask and washed with tetrahydrofuran (2×20 ml). The filtrate was distilled to a volume of 80 ml and to the concentrate was added n-propanol (110 ml). The mixture was distilled to a volume of 80 ml and the concentrate was used as is in the next step.

The concentrate was transferred to a 0.5 L multi-necked flask equipped with an agitator, temperature probe, condenser and nitrogen protection. The flask was charged with n-propanol (120 ml) followed by N-(3-Chloro-4-fluoro)phenyl-2-cyanoacetamide (12.4 g, 0.058 mole, 1.0 eq.). The mixture was heated to 75-80° C. The first of three portions of triethylorthoformate (8.6 g, 9.7 ml, 0.058 mole, d=0.89 g/ml) was added and the mixture heated to reflux (95-100° C.). After 2 hr, the second portion of triethylorthoformate was added (8.6 g, 9.7 ml, 0.058 mole). After another 2 hr, the third portion of triethylorthoformate was added (8.6 g, 9.7 ml, 0.058 mole). The mixture was held for a minimum of 20 hr at 95° C. The mixture was cooled to ambient temperature. The mixture was filtered on a Buchner funnel and washed with filtrate (20 ml) followed by chilled (0-10° C.) n-propanol (3×20 ml). The weight of the wet cake was −40 g.

The wet cake can be purified from acetonitrile. The wet cake was transferred to a 1-L multi-necked flask equipped with mechanical stirrer, condenser, temperature probe and nitrogen protection. The flask was charged with acetonitrile (390 ml), heated to 65-70° C. and held for a minimum of 20 min. The mixture was cooled to 60° C., filtered on a Buchner funnel and washed with acetonitrile (2×15 ml). The product was dried at 60° C., full vacuum for 20 h to give the titled compound (19.65 g, 67% overall yield over 2 steps). ¹H NMR: δ (DMSO-d₆) 11.43, (d, 1H, NHCH═C), 9.74, (s, 1H, NH), 8.56 (d, 1H, NHCH═C), 7.99-7.88 (m, 5H, Ar), 7.64-7.56 (m, 1H, Ar), 7.39-7.29 (m, 3H, Ar), 7.15-7.10 (m, 1H, Ar), 4.10 (q, 2H, OCH₂CH₃), 1.18 (t, 3H, OCH₂CH₃).

### Example 15

### Preparation of 3-cyano-4-(3-chloro-4-fluoroanilino)-7-ethoxy-N-phthalimidyl-quinoline.

To a 1-L multi-necked flask equipped with an agitator, temperature probe, condenser and nitrogen was charged 3-[3-ethoxy-4-(N-phthalimidyl)]anilino-N-(3-chloro-4-fluoro)phenyl-2-cyano-2-propenamide (18 g, 0.0356 mole) and suspended in acetonitrile (180 ml) and methanol (7.2 ml). A sodium hydroxide (40 g in 1 L water) scrubber system was set up. The mixture was heated to 65-70° C. and phosphorus oxychloride (54.6 g, 33.2 ml, 10.0 eq, d=1.645 g/ml) was added dropwise keeping the pot temperature at 65-70° C. The mixture eventually became thinner. The mixture was held for a minimum of 22 h and then cooled to 0-10° C. Water (90 ml) was added keeping the pot temperature <20° C. The pH of the mixture was adjusted to 8-10 using 28% ammonium hydroxide (−140 ml) keeping the pot temperature at 5-10° C. The 2-phase mixture was filtered on a Buchner funnel and washed with warm water. The solid was dried at 60° C., in a vacuum oven for 18 hr to give the titled compound (14.44 g, 83% uncorrected for strength) as an orange solid. ¹H NMR: δ (DMSO-d₆) 8.64 (s, 1H, Ar), 8.52 (m, 2H, Ar), 8.06-7.95 (m, 4H, phthalimide), 7.59-7.55 (m, 2H, Ar), 7.43 (m, 1H, Ar), 7.33 (m, 1H, Ar), 4.24 (q, 2H, OCH₂CH₃), 1.22 (t, 3H, OCH₂CH₃).

### Example 16

### Preparation of 6-amino-4-(3-chloro-4-fluoroanilino)-3-cyano-7-ethoxyquinoline

To a 500 ml multi-necked flask equipped with an agitator, temperature probe, condenser and nitrogen was charged 3-cyano-4-(3-chloro-4-fluoroanilino)-7-ethoxy-N-phthalimidyl-quinoline (12 g, 0.0246 mole) and suspended in ethanol. Next, 28% ammonium hydroxide (83 ml of a 7.4 M solution, 25 eq.) was added. The suspension was heated to 65-70° C. and held for a minimum of 2 hr. The reaction was cooled to room temperature, filtered on a Buchner funnel, and washed with water (3×15 ml) until the washes were colorless. The solid was dried at 60° C., in a vacuum oven for 25 hr to give the desired compound (4.13 g). A second crop (2.05 g) was obtained from acetonitrile recrystallization. (Overall yield 6.18 g, 70% uncorrected for strength). ¹H NMR: δ (DMSO-d₆) 9.22 (s, 1H, NH), 8.39 (s, 1H, Ar), 7.37-7.03 (m, 5H, Ar), 5.51 (s, 2H, NH₂), 4.24 (q, 2H, OCH₂CH₃), 1.45 (t, 3H, OCH₂CH₃).

Analytical Methods

NMR spectra were recorded on a Varian Inova 300 at 300 MHz (¹H and ¹³C) and chemical shifts were identified in ppm relative to TMS internal standard.

What is claimed is:

1. A method of preparing a substituted 3-cyanoquinoline comprising the step of treating an N-aryl-2-propene represented by formula III: 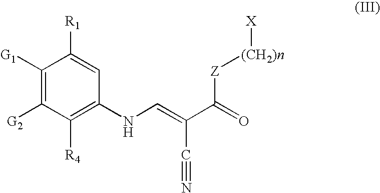
with POCl₃ to form a substituted 3-cyanoquinoline represented by formula XI: 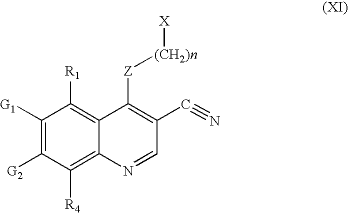
wherein:
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono-di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
or
X is a radical having the formula: 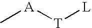
wherein
A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR —(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂ NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z is —NH—, —O—, —S—, or —NR—;
R is alkyl of 1-6 carbon atoms, or carboalkyl of 2-7 carbon atoms;
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 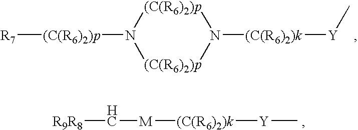
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₁ and/or G₂ are independently selected from a protected amino group and R₂—NH—;
or if any of the substituents R₁, G₁, G₂, or R₄ are located on contiguous carbon atoms then they may be taken together as the divalent radical —O—C(R₆) 2-O—;
Y is a divalent radical selected from the group consisting of 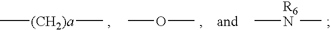
R₇ is —NR₆R₇, —OR₆, -J, —N(R₆)₃⁺, or —NR₇(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 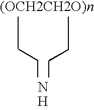
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or —(C(R₆)₂) 5 N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 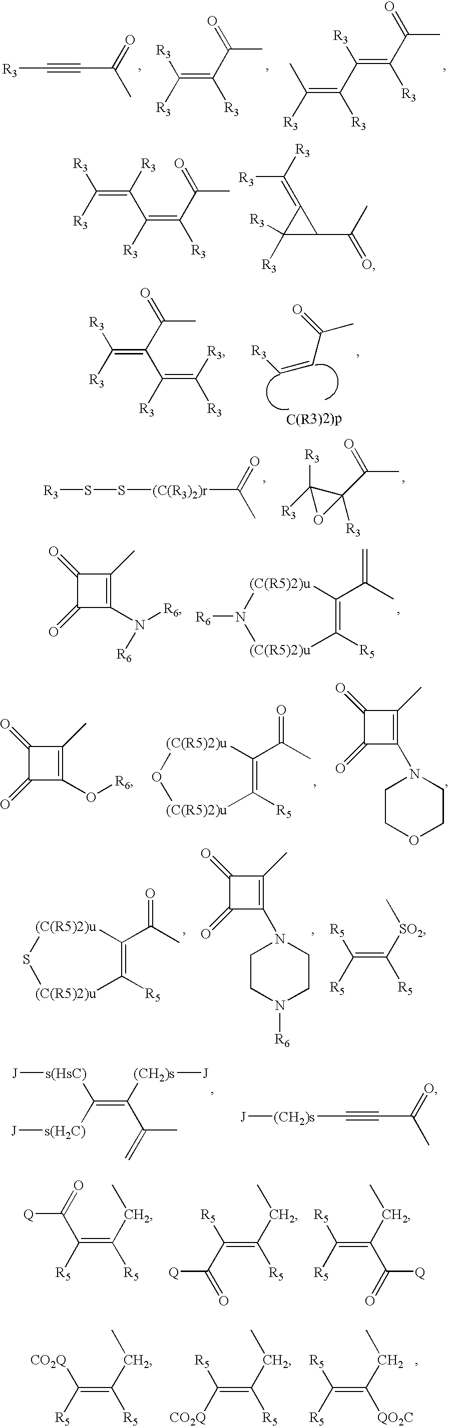
R₃ is independently hydrogen, alkyl of 1-6 carbon atoms, carboxy, carboalkoxy of 1-6 carbon atoms, phenyl, carboalkyl of 2-7 carbon atoms, 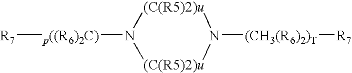
R₇—(C(R₆)₂)ₛ—, R₇—(C(R₆)₂)ₚ-M-(C(R₇)₂)ᵣ—, R₈R₉—CH-M-(C(R₆)₂)ᵣ—, Het-(C(R₆)₂)_{q}—W—(C(R₆)₂)ᵣ—; aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms;
R₅ is independently hydrogen, alkyl of 1-6 carbon atoms, carboxy, carboalkoxy of 1-6 carbon atoms, phenyl, carboalkyl of 2-7 carbon atoms, 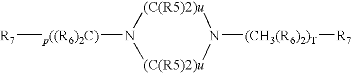
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣ NR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 24;
or a pharmaceutically acceptable salt thereof, provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
when Y is —NR₆— and R₇ is —NR₆R₆—N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4, when W is a bond with Het bonded through a nitrogen atom and Y is —O—or —NR₆— then k=2-4.

2. The method according to claim 1, further comprising the step of forming the N-aryl-2-propene compound of formula III by condensing an N-arylformimidate of formula I: 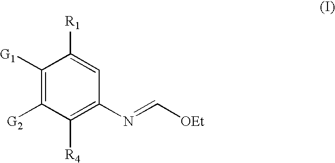
with an active methylene of formula XII:

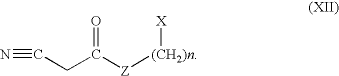

3. The method according to claim 2, further comprising the step of forming the N-arylformimidate of formula I by reacting an arylamine of formula XIII: 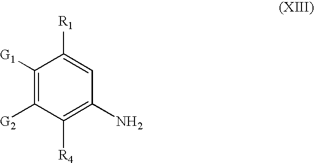
with CH(OEt)₃.

4. The method according to claim 1 further comprising the step of forming the N-aryl-2-propene of formula III by reacting an alkoxymethylene derivative of formula II: 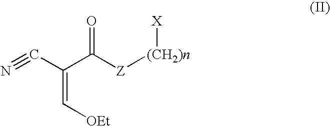
with an arylamine of formula XIII:

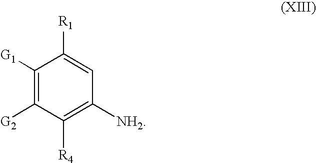

5. The method according to claim 4, further comprising the step of forming an alkoxymethylene of formula II by condensing an active methylene of formula XII: 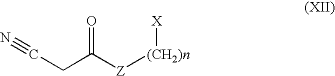
with CH(OEt)₃.

6. The method according to claims 1, 2, 3, 4, and 5, wherein Z is selected from the group consisting of NH, O and S.

7. The method according to claim 1, 2, 3, 4, and 5, wherein G₁ is a protected amino group selected from the group consisting of acetamides, benzamides, cyclic imides, pyrroles, tert-butoxycarbonyl amine and benzyloxycarbonyl amide.

8. The method according to claim 7, wherein G₁ is phthalimide.

9. A method according to claims 1, 2, 3, 4, and 5, wherein X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono- di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino.

10. The method according to claims 1, 2, 3, 4, and 5, wherein the N-aryl-2-propene of formula III is the compound represented by formula IX:

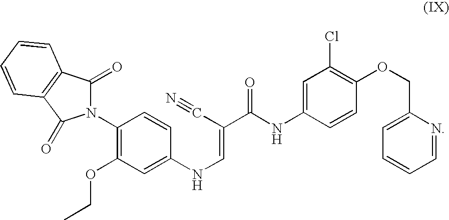

11. The method according to claims 2 and 3, wherein the N-arylformimidate of formula I is a compound represented by formula IV: 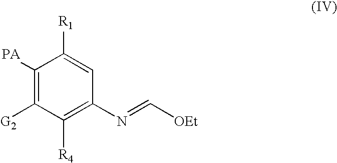
wherein PA is a protected amino group.

12. The method according to claim 11, wherein the N-arylformimidate of formula IV is a compound represented by formula VII:

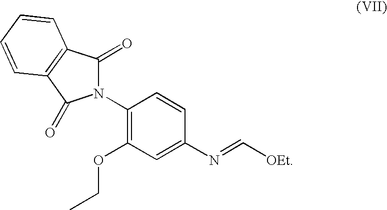

13. The method according to claims 3, 4 and 5, wherein the arylamine of formula XIII is a compound represented by formula XVI:

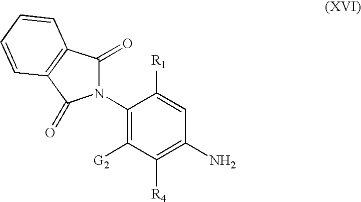

14. The method according to claim 13, wherein the arylamine of formula XVI is a compound represented by formula XIV:

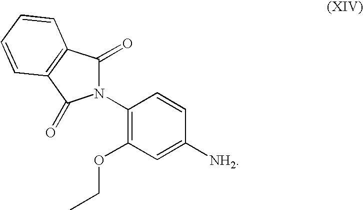

15. The method according to claims 4 and 5, wherein the alkoxymethylene of formula II is a compound represented by formula VIII:

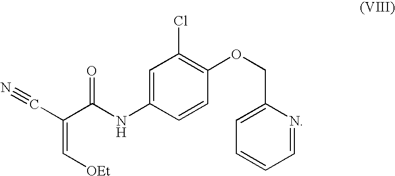

16. The method according to claims 2, 3 and 5, wherein the active methylene of formula XII is the compound represented by formula XV:

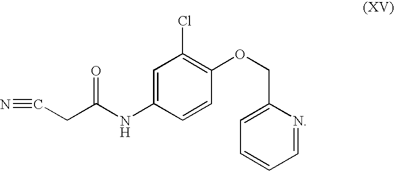

17. A compound represented by formula I: 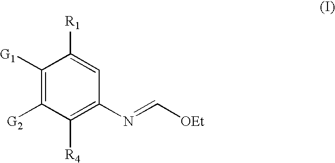
wherein
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 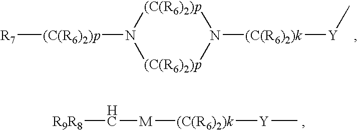
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₁ and/or G₂ are independently selected from a protected amino group and R₂—NH—;
or if any of the substituents R₁, G₁, G₂, or R₄ are located on contiguous carbon atoms then they may be taken together as the divalent radical —O—C(R₆) 2-O—;
Y is a divalent radical selected from the group consisting of 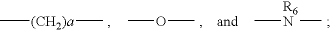
R₇ is —NR₆R₇, —OR₆, -J, —N(R₆)₃⁺, or —NR₇6(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 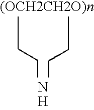
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or
—(C(R₆)₂), N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 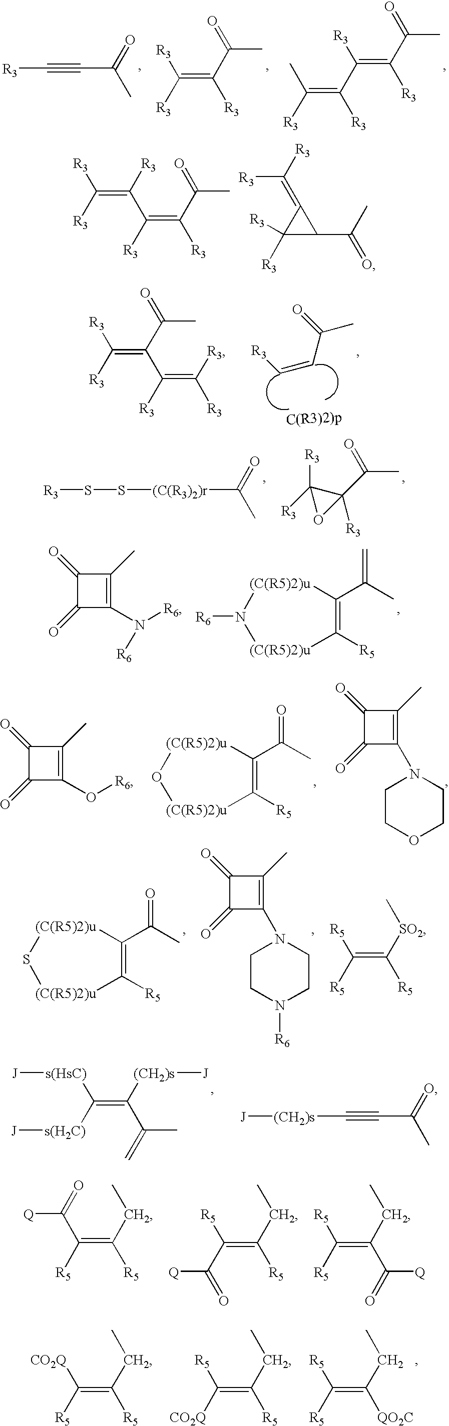
R₃ is independently hydrogen, alkyl of 1-6 carbon atoms, carboxy, carboalkoxy of 1-6 carbon atoms, phenyl, carboalkyl of 2-7 carbon atoms, 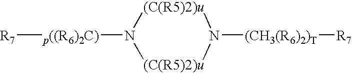
R₇—(C(R₆)₂)ₛ—, R₇—(C(R₆)₂)ₚ-M-(C(R₇)₂)ᵣ—, R₈R₉—CH-M-(C(R₆)₂)ᵣ—, Het-(C(R₆)₂)_{q}—W—(C(R₆)₂)ᵣ—; aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms;
R₅ is independently hydrogen, alkyl of 1-6 carbon atoms, carboxy, carboalkoxy of 1-6 carbon atoms, phenyl, carboalkyl of 2-7 carbon atoms, 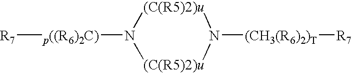
R₇—(C(R₆)₂)ₛ—, R₇—(C(R₆)₂)ₚ-M-(C(R₆)₂)ᵣ—, R₈R₉—CH-M-(C(R₆)₂) r- or Het-(C(R₆)₂)_{q}—W—(C(R₆)₂)ᵣ—;
R₈, and R₉ are each, independently, —(C(R₆)₂)ᵣ NR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v-4, wherein the sum of u+v is 2-4;
or a pharmaceutically acceptable salt thereof, provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
when Y is —NR₆— and R₇ is —NR₆R₆—N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4, and when W is a bond with Het bonded through a nitrogen atom and Y is —O— or —NR₆— then k=2-4.

18. A compound represented by formula II: 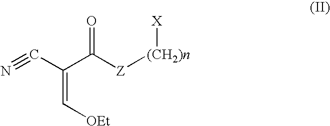
wherein:
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono-di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
or
X is a radical having the formula: 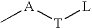
wherein
A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR —(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂ NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z is —NH—, —O—, —S—, or —NR—; and
R is alkyl of 1-6 carbon atoms, or carboalkyl of 2-7 carbon atoms.

19. A compound represented by formula III: 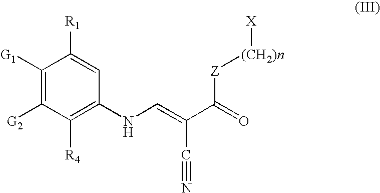
wherein:
X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono-di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
or
X is a radical having the formula: 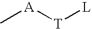
wherein
A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR —(CH₂)ₘ—, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂ NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z is —NH—, —O—, —S—, or —NR—;
R is alkyl of 1-6 carbon atoms, or carboalkyl of 2-7 carbon atoms;
G₁, G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 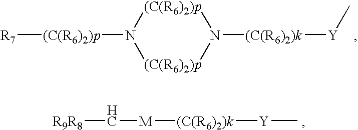
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₁ and/or G₂ are independently selected from a protected amino group and R₂—NH—;
or if any of the substituents R₁, G₁, G₂, or R₄ are located on contiguous carbon atoms then they may be taken together as the divalent radical —O—C(R₆)₂—O—;
Y is a divalent radical selected from the group consisting of 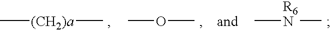
R₇ is —NR₆R₇, —OR₆, -J, —N(R₆)₃⁺, or —NR₇6(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 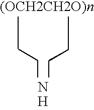
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or
—(C(R₆)₂)ₛ N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 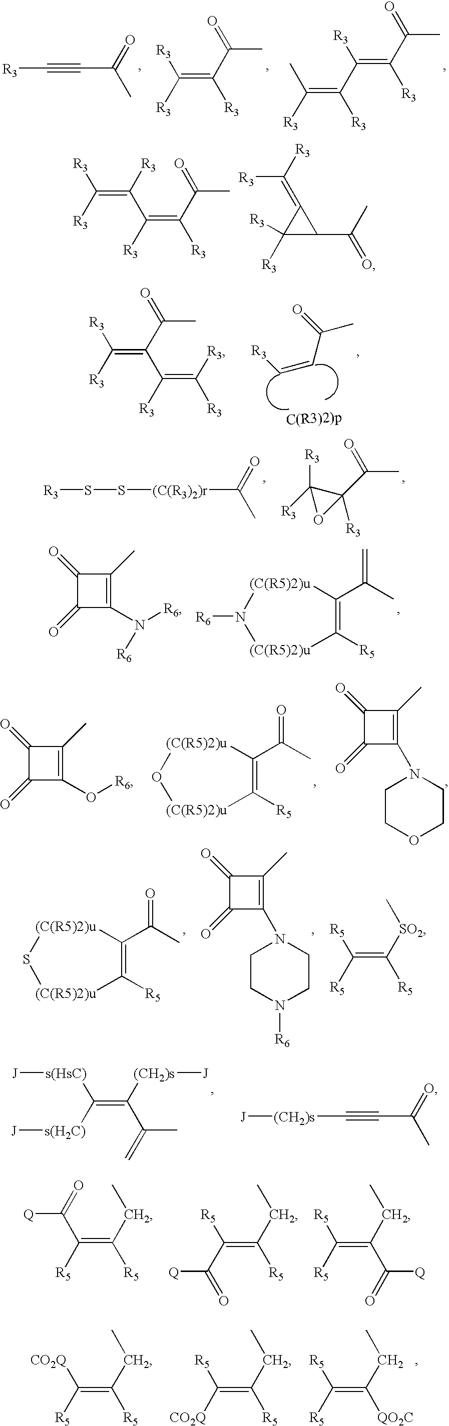
R₃ is independently hydrogen, alkyl of 1-6 carbon atoms, carboxy, carboalkoxy of 1-6 carbon atoms, phenyl, carboalkyl of 2-7 carbon atoms, 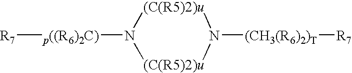
R₇—(C(R₆)₂)ₛ—, R₇—(C(R₆)₂)ₚ-M-(C(R₇)₂)ᵣ—, R₈R₉—CH-M-(C(R₆)₂)ᵣ—, Het-(C(R₆)₂)_{q}—W—(C(R₆)₂)ᵣ—; aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms;
R₅ is independently hydrogen, alkyl of 1-6 carbon atoms, carboxy, carboalkoxy of 1-6 carbon atoms, phenyl, carboalkyl of 2-7 carbon atoms, 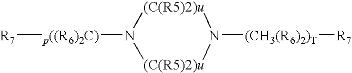
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣ NR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
or a pharmaceutically acceptable salt thereof, provided that when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that when Y is —NR₆— and R₇ is —NR₆R₆—N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4 and when W is a bond with Het bonded through a nitrogen atom and Y is —O— or —NR₆— then k=2-4.

20. Compound represented by formula IV: 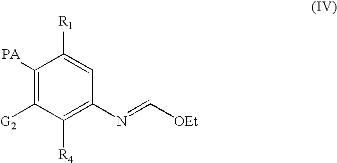
wherein PA is a protected amino group;
wherein G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 14 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 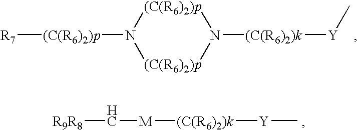
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₂ is selected from a protected amino group and R₂—NH—;
Y is a divalent radical selected from the group consisting of 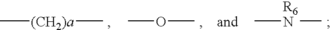
R₇ is —NR₆R₇, —OR₆, -J, —N(R₆)₃⁺, or —NR₇6(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 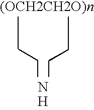
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or —(C(R₆)₂)ₛ N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 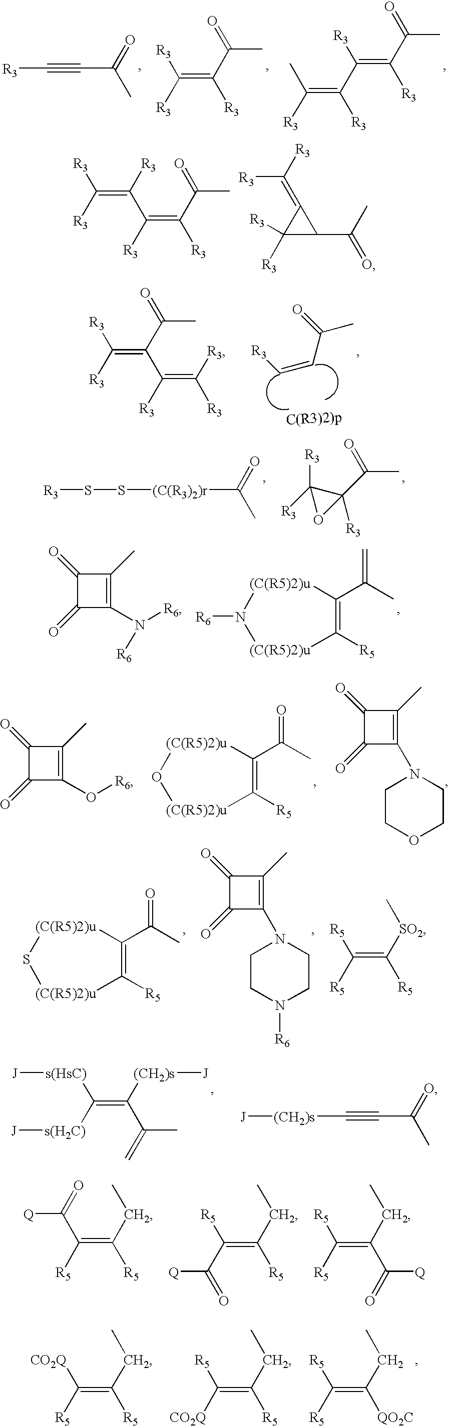
R₃ is independently hydrogen, alkyl of 1-6 carbon atoms, carboxy, carboalkoxy of 1-6 carbon atoms, phenyl, carboalkyl of 2-7 carbon atoms, 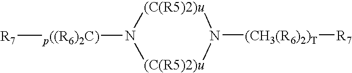
R₇—(C(R₆)₂)ₛ—, R₇—(C(R₆)₂)ₚ-M-(C(R₇)₂)ᵣ—, R₈R₉—CH-M-(C(R₆)₂)ᵣ—, Het-(C(R₆)₂)_{q}—W—(C(R₆)₂)ᵣ—; aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms;
R₅ is independently hydrogen, alkyl of 1-6 carbon atoms, carboxy, carboalkoxy of 1-6 carbon atoms, phenyl, carboalkyl of 2-7 carbon atoms, 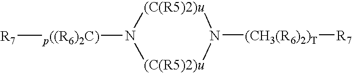
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣ NR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4; n is 0-1;
m is 0-3; p=2-4;
q=0-4;
r=1-4;
s=1-6; u=04 and v=0-4, wherein the sum of u+v is 2-4;
or a pharmaceutically acceptable salt thereof, provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
when Y is —NR₆— and R₇ is —NR₆R₆—N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4, and when W is a bond with Het bonded through a nitrogen atom and Y is —O— or —NR₆— then k=2-4.

21. A compound represented by formula V: 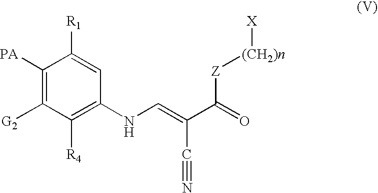
wherein PA is a protected amino group;
wherein X is a bicyclic aryl or bicyclic heteroaryl ring system of 8 to 12 atoms where the bicyclic heteroaryl ring contains 1 to 4 heteroatoms selected from N, O, and S with the proviso that the bicyclic heteroaryl ring does not contain O—O, S—S, or S—O bonds and where the bicyclic aryl or bicyclic heteroaryl ring may be optionally mono- di-, tri, or tetra-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkyl amino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; or
X is cycloalkyl of 3 to 7 carbon atoms, which may be optionally substituted with one or more alkyl of 1 to 6 carbon atom groups; or is a pyridinyl, pyrimidinyl, or phenyl ring; wherein the pyridinyl, pyrimidinyl, or phenyl ring optionally mono-di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, and benzoylamino;
or
X is a radical having the formula: 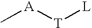
wherein
A is a pyridinyl, pyrimidinyl, or phenyl ring; wherein the pyridinyl, pyrimidinyl, or phenyl ring may be optionally mono- or di-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
T is bonded to a carbon of A and is:
—NH(CH₂)ₘ—, —O(CH₂)ₘ—, —S(CH₂)ₘ—, —NR —(CH₂)ₘ, —(CH₂)ₘ—, —(CH₂)ₘNH—, —(CH₂)ₘO—, —(CH₂)ₘS—, or —(CH₂)ₘNR—;
L is an unsubsitituted phenyl ring or a phenyl ring mono-, di-, or tri-substituted with a substituent selected from the group consisting of halogen, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino; provided that L can be an unsubstituted phenyl ring only when m>0 and T is not —CH₂ NH— or —CH₂O—; or
L is a 5- or 6-membered heteroaryl ring where the heteroaryl ring contains 1 to 3 heteroatoms selected from N, O, and S, with the proviso that the heteroaryl ring does not contain O—O, S—S, or S—O bonds, and where the heteroaryl ring is optionally mono- or di-substituted with a substituent selected from the group consisting of halogen, oxo, thio, alkyl of 1-6 carbon atoms, alkenyl of 2-6 carbon atoms, alkynyl of 2-6 carbon atoms, azido, hydroxyalkyl of 1-6 carbon atoms, halomethyl, alkoxymethyl of 2-7 carbon atoms, alkanoyloxymethyl of 2-7 carbon atoms, alkoxy of 1-6 carbon atoms, alkylthio of 1-6 carbon atoms, hydroxy, trifluoromethyl, cyano, nitro, carboxy, carboalkoxy of 2-7 carbon atoms, carboalkyl of 2-7 carbon atoms, phenoxy, phenyl, thiophenoxy, benzoyl, benzyl, amino, alkylamino of 1-6 carbon atoms, dialkylamino of 2 to 12 carbon atoms, phenylamino, benzylamino, alkanoylamino of 1-6 carbon atoms, alkenoylamino of 3-8 carbon atoms, alkynoylamino of 3-8 carbon atoms, carboxyalkyl of 2-7 carbon atoms, carboalkoxyalkyl of 3-8 carbon atoms, aminoalkyl of 1-5 carbon atoms, N-alkylaminoalkyl of 2-9 carbon atoms, N,N-dialkylaminoalkyl of 3-10 carbon atoms, N-alkylaminoalkoxy of 2-9 carbon atoms, N,N-dialkylaminoalkoxy of 3-10 carbon atoms, mercapto, and benzoylamino;
Z is —NH—, —O—, —S—, or —NR—;
R is alkyl of 1-6 carbon atoms, or carboalkyl of 2-7 carbon atoms;
G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 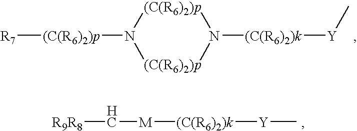
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₂ is selected from a protected amino group and R₂—NH—;
Y is a divalent radical selected from the group consisting of 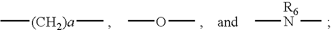
R₇ is —NR₆R₇, —OR₆, -J, —N(R₆)₃⁺, or —NR₇6(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 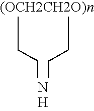
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or —(C(R₆)₂)ₛ N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 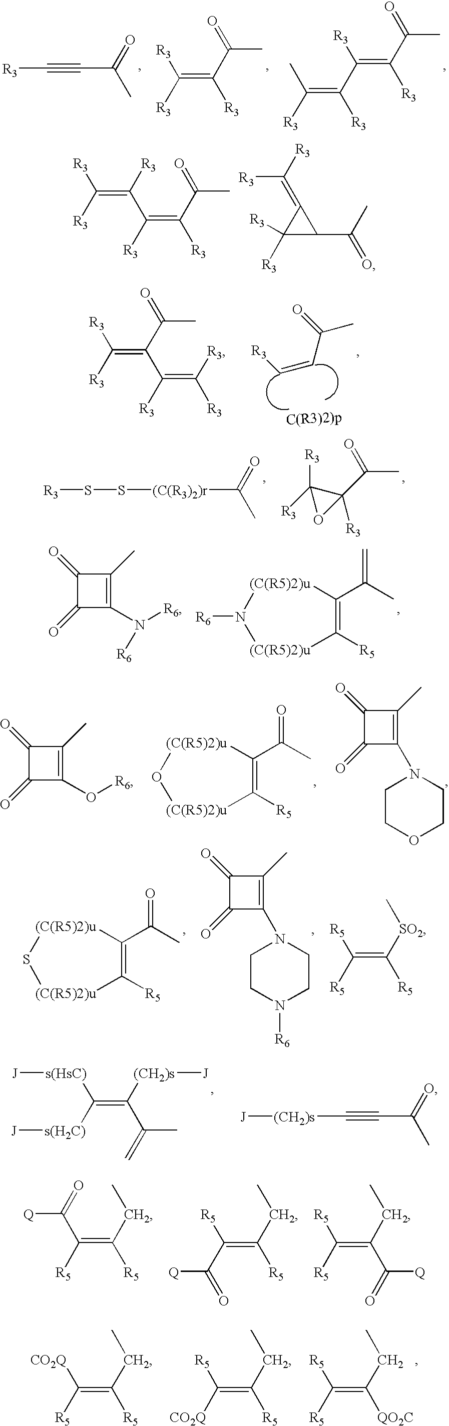
R₃ is independently hydrogen, alkyl of 1-6 carbon atoms, carboxy, carboalkoxy of 1-6 carbon atoms, phenyl, carboalkyl of 2-7 carbon atoms, 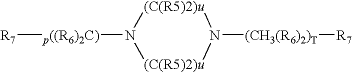
R₇—(C(R₆)₂)ₛ—, R₇—(C(R₆)₂)ₚ-M-(C(R₇)₂)ᵣ—, R₈R₉—CH-M-(C(R₆)₂)ᵣ—, Het-(C(R₆)₂)_{q}—W—(C(R₆)₂)ᵣ—; aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms;
R₅ is independently hydrogen, alkyl of 1-6 carbon atoms, carboxy, carboalkoxy of 1-6 carbon atoms, phenyl, carboalkyl of 2-7 carbon atoms, 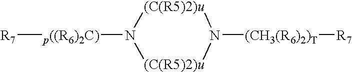
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆)₂)ᵣ—;
R₈, and R₉ are each, independently, —(C(R₆)₂)ᵣ NR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4; n is 0-1;
m is 0-3;
p=2-4;
q=0-4;
r=1-4;
s=1-6;
u=0-4 and v=0-4, wherein the sum of u+v is 2-4;
or a pharmaceutically acceptable salt thereof, provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
when Y is —NR₆— and R₇ is —NR₆R₆—N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4, and when W is a bond with Het bonded through a nitrogen atom and Y is —O— or —NR₆— then k=2-4

22. A compound represented by formula XVI: 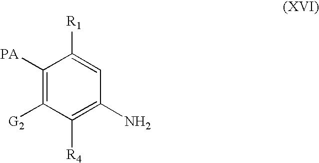
wherein PA is a protected amino group;
G₂, R₁, and R₄ are each, independently, hydrogen, halogen, alkyl of 1-6 carbon atoms, alkenyl of 2-6 carbon atoms, alkynyl of 2-6 carbon atoms, alkenyloxy of 2-6 carbon atoms, alkynyloxy of 2-6 carbon atoms, hydroxymethyl, halomethyl, alkanoyloxy of 1-6 carbon atoms, alkenoyloxy of 3-8 carbon atoms, alkynoyloxy of 3-8 carbon atoms, alkanoyloxymethyl of 2-7 carbon atoms, alkenoyloxymethyl of 4-9 carbon atoms, alkynoyloxymethyl of 4-9 carbon atoms, alkoxymethyl of 2-7 carbon atoms, alkoxy of 1-6 carbon atoms, alkylthio of 1-6 carbon atoms, alkylsulphinyl of 1-6 carbon atoms, alkylsulphonyl of 1-6 carbon atoms, alkylsulfonamido of 1-6 carbon atoms, alkenylsulfonamido of 2-6 carbon atoms, alkynylsulfonamido of 2-6 carbon atoms, hydroxy, trifluoromethyl, trifluoromethoxy, cyano, nitro, carboxy, carboalkoxy of 2-7 carbon atoms, carboalkyl of 2-7 carbon atoms, phenoxy, phthalimide, phenyl, thiophenoxy, benzyl, amino, hydroxyamino, alkoxyamino of 1-4 carbon atoms, alkylamino of 1-6 carbon atoms, dialkylamino of 2 to 12 carbon atoms, N-alkylcarbamoyl, N,N-dialkylcarbamoyl, N-alkyl-N-alkenylamino of 4 to 12 carbon atoms, N,N-dialkenylamino of 6-12 carbon atoms, phenylamino, benzylamino, 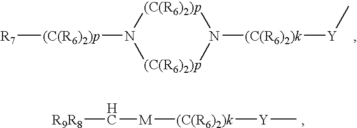
R₇—(C(R₆)₂)_{g}—Y—, R₇—(C(R₆)₂)ₚ-M-(C(R₆)₂)ₖ—Y—, or Het-(C(R₆)₂)_{q}W—(C(R₆)₂—Y—; or optionally G₂ is selected from a protected amino group and R₂—NH—;
Y is a divalent radical selected from the group consisting of 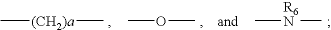
R₇ is —NR₆R₇, —OR₆, -J, —N(R₆)₃⁺, or —NR₇6(OR₆);
M is >NR₆, —O—, >N—(C(R₆)₂)ₚ NR₆R₆, or >N—(C(R₆)₂)ₚ—OR₆;
W is >NR₆, —O— or is a bond;
Het is selected from the group consisting of morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane, tetrahydropyran, and 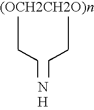
wherein Het is optionally mono- or di-substituted on carbon or nitrogen with R₆, optionally mono- or di-substituted on carbon with hydroxy, —N(₆)₂, or —OR₆, optionally mono or di-substituted on carbon with the mono-valent radicals —(C(R₆)₂)ₛOR₆ or —(C(R₆)₂)ₛ N(R₆)₂, and optionally mono or di-substituted on a saturated carbon with divalent radicals —O— or —O(C(R₆)₂)ₛO—;
R₆ is hydrogen, alkyl of 1-6 carbon atoms, alkenyl of 2-6 carbon atoms, alkynyl of 2-6 carbon atoms, cycloalkyl of 1-6 carbon atoms, carboalkyl of 2-7 carbon atoms, carboxyalkyl (2-7 carbon atoms), phenyl, or phenyl optionally substituted with one or more halogen, alkoxy of 1-6 carbon atoms, trifluoromethyl, amino, alkylamino of 1-3 carbon atoms, dialkylamino of 2-6 carbon atoms, nitro, cyano, azido, halomethyl, alkoxymethyl of 2-7 carbon atoms, alkanoyloxymethyl of 2-7 carbon atoms, alkylthio of 1-6 carbon atoms, hydroxy, carboxyl, carboalkoxy of 2-7 carbon atoms, phenoxy, phenyl, thiophenoxy, benzoyl, benzyl, phenylamino, benzylamino, alkanoylamino of 1-6 carbon atoms, or alkyl of 1-6 carbon atoms; with the proviso that the alkenyl or alkynyl moiety is bound to a nitrogen or oxygen atom through a saturated carbon atom;
R₂, is selected from the group consisting of 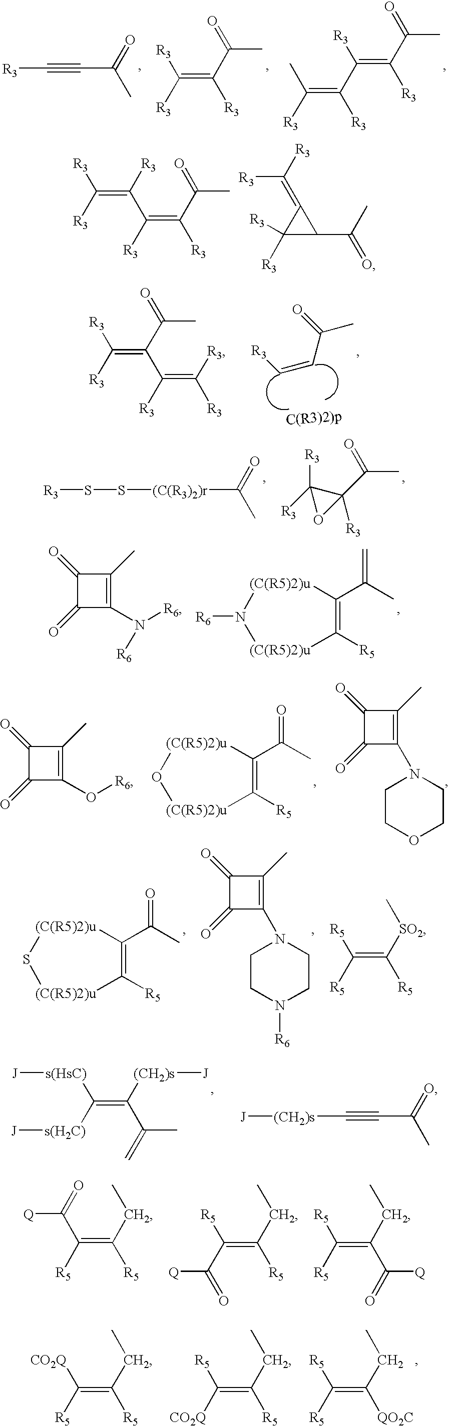
R₃ is independently hydrogen, alkyl of 1-6 carbon atoms, carboxy, carboalkoxy of 1-6 carbon atoms, phenyl, carboalkyl of 2-7 carbon atoms, 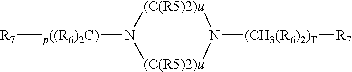
R₇—(C(R₆)₂)ₛ—, R₇—(C(R₆)₂)ₚ-M-(C(R₇)₂)ᵣ—, R₈R₉—CH-M-(C(R₆)₂)ᵣ—, Het-(C(R₆)₂)_{q}—W—(C(R₆)₂)ᵣ—; aminoalkyl of 1-6 carbon atoms, N-alkylaminoalkyl of 2-9 carbon atoms, N,N-dialkylaminoalkyl of 3-12 carbon atoms, N-cycloalkylaminoalkyl of 4-12 carbon atoms, N-cycloalkyl-N-alkylaminoalkyl of 5-18 carbon atoms, N,N-dicycloalkylaminoalkyl of 7-18 carbon atoms, morpholino-N-alkyl wherein the alkyl group is 1-6 carbon atoms, piperidino-N-alkyl wherein the alkyl group is 1-6 carbon atoms, N-alkyl-piperidino-N-alkyl wherein either alkyl group is 1-6 carbon atoms;
R₅ is independently hydrogen, alkyl of 1-6 carbon atoms, carboxy, carboalkoxy of 1-6 carbon atoms, phenyl, carboalkyl of 2-7 carbon atoms, 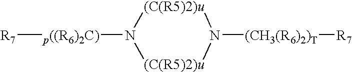
R₇—(C(R₆)₂)ₛ—, R₇—(C(R₆)₂)ₚ-M-(C(R₆)₂)ᵣ—, R₈R₉—CH-M-(C(R₆)₂)ᵣ—, or Het-(C(R₆)₂)_{q}—W—(C(R₆) 2)ᵣ—;
R₈, and R₉ are each, independently, —(C(R₆)₂)ᵣ NR₆R₆, or —(C(R₆)₂)ᵣOR₆;
J is independently hydrogen, chlorine, fluorine, or bromine;
Q is alkyl of 1-6 carbon atoms or hydrogen;
a=0 or 1;
g=1-6;
k=0-4;
n is 0-1;
m is 0-3;
p=2-4;
q=0-4;
r=1-4;
s=1-6;
u=0-4 and v=0-4, wherein the sum of u+v is 2-4;
or a pharmaceutically acceptable salt thereof, provided that
when R₆ is alkenyl of 2-7 carbon atoms or alkynyl of 2-7 carbon atoms, such alkenyl or alkynyl moiety is bound to a nitrogen or oxygen atom through a saturated carbon atom;
and further provided that
when Y is —NR₆— and R₇ is —NR₆R₆—N(R₆)₃⁺, or —NR₆(OR₆), then g=2-6;
when M is —O— and R₇ is —OR₆ then p=1-4;
when Y is —NR₆— then k=2-4;
when Y is —O— and M or W is —O— then k=1-4
when W is not a bond with Het bonded through a nitrogen atom then q=2-4, and when W is a bond with Het bonded through a nitrogen atom and Y is —O— or —NR₆— then k=2-4.

23. The compound according to claims 20, 21 and 22, wherein the protected amino group is selected from the group consisting of acetamides, benzamides, cyclic imides, pyrroles, tert-butoxycarbonyl amine and benzyloxycarbonyl amide.

24. The compound according to claim 23, wherein the protected amino group is phthalimide.

25. A compound represented by formula VII:

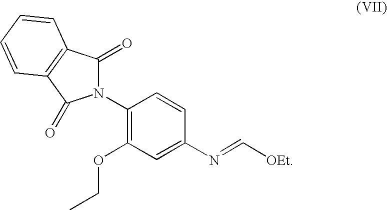

26. A compound represented by formula VIII:

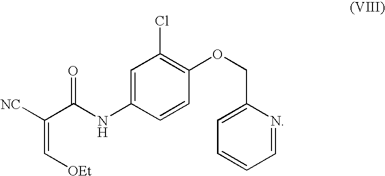

27. A compound represented by formula IX:

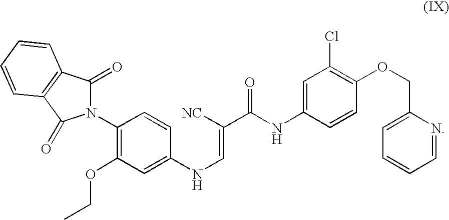

28. The method according to claim 1, 2, 3, 4, and 5, wherein G₁ is a halogen selected from the group consisting of F, Cl, Br, or I.

29. The method according to claims 1, 2, 3, 4, and 5, wherein the N-aryl-2-propene of formula III is the compound represented by formula IX′:

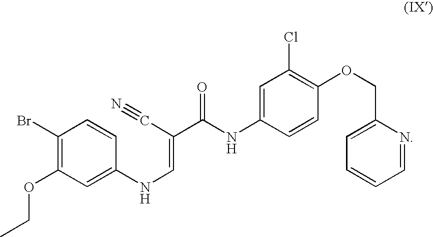

30. The method according to claims 1, 2, 3, 4 and 5, wherein the N-arylformimidate of formula I is a compound represented by formula VII′:

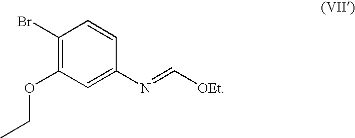

31. The method according to claims 3, 4 and 5, wherein the arylamine of formula XIII is a compound represented by the structure:

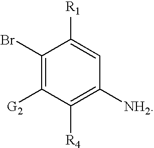

32. The method according to claim 31, wherein the arylamine is:

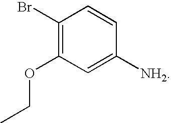

33. A compound of formula VII′:

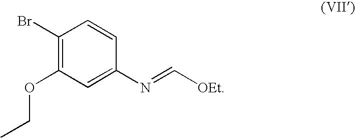

34. A compound of formula IX′: [chemical structure]
